(19) United States
(12) Patent Application Publication
(10) Pub. No.: US 2020/0129566 A1
(43) Pub. Date: Apr. 30, 2020

(54) METHOD FOR DETERMINING THE POTENTIAL EFFICACY OF ANTICANCER TREATMENT

(71) Applicants: ASSISTANCE PUBLIQUE - HOPITAUX DE PARIS, Paris (FR); INSTITUT NATIONAL DE LA RECHERCHE AGRONOMIQUE, Paris (FR); UNIVERSITE PARIS-SUD, Orsay (FR); INSTITUT GUSTAVE ROUSSY, Villejuif (FR)

(72) Inventor: Franck Carbonnel, Paris (FR)

(73) Assignees: ASSISTANCE PUBLIQUE - HOPITAUX DE PARIS, Paris (FR); INSTITUT NATIONAL DE LA RECHERCHE AGRONOMIQUE, Paris (FR); UNIVERSITE PARIS-SUD, Orsay (FR); INSTITUT GUSTAVE ROUSSY, Villejuif (FR)

(21) Appl. No.: 16/494,095

(22) PCT Filed: Mar. 22, 2018
(86) PCT No.: PCT/EP2018/057361

§ 371 Date: Sep. 13, 2019

(30) Foreign Application Priority Data
Mar. 22, 2017 (EP) 17305327.3

(51) Int. Cl. A61K 35/74; G01N 33/574; A61K 9/00

(52) U.S. Cl. CPC A61K 35/74; G01N 33/5743; G01N 2333/70521; A61K 9/0065; A61K 9/0056

(57) ABSTRACT

Embodiments of the present disclosure relate to methods for ex vivo determining whether a patient with metastatic melanoma is likely to benefit from a treatment with an anti CTLA-4 molecule, preferably ipilimumab, by analyzing the gut microbiota in a fecal sample from said patient.

The present invention relates to the field of anticancer treatment, more specifically to methods for ex vivo determining whether a patient with metastatic melanoma is likely to benefit from a treatment with an anti CTLA-4 molecule, preferably ipilimumab, by analysing the gut microbiota in a fecal sample from said patient.

Immunotherapy has become a major therapeutic strategy in oncology. The trail was blazed by an antibody directed against Cytotoxic T-lymphocyte-associated antigen 4 (CTLA-4), namely ipilimumab, which demonstrated a significant survival benefit in patients with metastatic melanoma (MM).^{1,2} It has previously been reported that 22% of MM patients treated with ipilimumab have a prolonged survival.³

CTLA-4 is a homolog of CD28; it down regulates T cell costimulation during antigen presentation, and thus constitutes a non-specific negative checkpoint of the immune response.⁴⁻⁶ Ipilimumab reactivates T cells that are primed in lymphoid organs, regardless of the antigen specificity, and therefore affects a much wider repertoire of T cells than those involved in the anti-cancer response.^{7,8} This probably explains why CTLA-4 blockade is associated with numerous and various immune-related adverse events (irAE). These irAE mainly affect the skin, the gut, the endocrine glands and the liver.^{9,10} Colitis affects 8 to 22% of patients treated with ipilimumab^{10,11} and requires discontinuation of ipilimumab and high dose steroids. Some patients with severe colitis may need anti-TNF therapy and sometimes colectomy.^{11,12} In summary, ipilimumab benefits a limited percentage of patients and can cause potentially severe irAE. In this context, reliable biomarkers of efficacy and/or toxicity capable of optimizing the risk/benefit ratio of this drug would be of paramount interest.

Colitis due to ipilimumab shares several features with inflammatory bowel disease (IBD).¹² In IBD, deregulated inflammation is associated with a dysbiotic composition of the intestinal microbiota, which is supposed to be the antigenic drive of the disease.¹³⁻¹⁵ The trillions of bacteria that constitute the human gut microbiota participate in the proper maturation of both mucosal and systemic immune system, and it has long been known that the composition of the vertebrates' microbiome was mandatory for the promotion of an appropriate immune response against pathogens and the maintenance of immune homeostasis.¹⁶ In addition, studies performed in mice have suggested that the microbiome composition was critical to promote an anti-tumor immune response to anti CTLA-4 and anti PD-1 monoclonal antibodies.^{17,18}

Inventors have conducted prospective study on patients with metastatic melanoma treated with ipilimumab. Fecal microbiota composition was assessed at baseline and before each ipilimumab infusion. Microbiota was studied using 16S rRNA gene sequencing; patients were clustered based on dominant microbiota pattern.

Based on this study, Inventors have now demonstrated that anticancer response as well as immune-related enterocolitis due to ipilimumab depend on microbiome composition.

Indeed results presented in the experimental part show that a distinct baseline gut microbiota composition was associated with both clinical response and colitis. As compared to patients whose baseline microbiota was driven by Bacteroides (cluster B, n=12), patients whose baseline microbiota was enriched with Faecalibacterium genus and other Firmicutes (cluster A, n=10) had longer progression-free survival (p=0.0039) and overall survival (p=0.051). Most of the baseline colitis-associated phylotypes were related to Firmicutes (eg, relatives of Faecalibacterium prausnitzii and Gemmiger formicilis), whereas no-colitis related phylotypes were assigned to Bacteroidetes. A low proportion of peripheral blood regulatory T cells was associated with cluster A, long-term clinical benefit and colitis. Ipilimumab led to a higher Inducible T-cell COStimulator induction on CD4+ T cells and to a higher increase in serum CD25 in patients who belonged to cluster A.

Baseline gut microbiota enriched with Faecalibacterium and other Firmicutes is associated with beneficial clinical response to ipilimumab and more frequent occurrence of ipilimumab-associated colitis.

The present invention thus relates to a method for predicting the response of an individual to an anticancer treatment with anti-CTL4 molecules comprising the steps of:

(i) determining gut microbial OTU (or phylotype or molecular species; i.e any group of 16S rRNA gene sequences sharing at least 97% of similarity between each other and hence grouped together in a taxonomic entity called an OTU: Operational Taxonomic Unit; and represented by a selected representative sequence) in a fecal sample of said individual;

(ii) determining a relative abundance of at least one OTU comprising a nucleotide fragment of sequence having at least 97%, preferably 98%, 99% or 100%, identity with a nucleic sequence selected in the group consisting of: denovo3066 of SEQ. ID. No1, denovo991 of SEQ. ID. No2, denovo4666 of SEQ. ID. No3, denovo5905 of SEQ. ID. No4, denovo3795 of SEQ. ID. No5, denovo4787 of SEQ. ID. No6, denovo453 of SEQ. ID. No7, denovo4054 of SEQ. ID. No8, denovo3816 of SEQ. ID. No9, denovo3657 of SEQ. ID. No10, denovo5800 of SEQ. ID. No11 and denovo5178 of SEQ. ID. No12; preferably, determining a relative abundance of at least one OTU comprising a nucleotide fragment of sequence having at least 97%, preferably 98%, 99% or 100%, identity with a nucleic sequence selected in the group consisting of: denovo3066 of SEQ. ID. No1, denovo991 of SEQ. ID. No2, denovo3795 of SEQ. ID. No5, denovo4787 of SEQ. ID. No6, denovo4054 of SEQ. ID. No8, denovo3816 of SEQ. ID. No9, denovo3657 of SEQ. ID. No10 and denovo5178 of SEQ. ID. No12; wherein individual having a gut microbiote enriched in at least one OTU selected in the group consisting of denovo3066 of SEQ. ID. No1, denovo991 of SEQ. ID. No2, denovo4666 of SEQ. ID. No3, denovo5905 of SEQ. ID. No4, denovo3795 of SEQ. ID. No5, denovo4787 of SEQ. ID. No6, denovo453 of SEQ. ID. No7 and denovo5178 of SEQ. ID. No12 and/or depleted in at least one OTU selected in the group consisting of denovo4054 of SEQ. ID. No8, denovo3816 of SEQ. ID. No9, denovo3657 of SEQ. ID. No10 and denovo5800 of SEQ. ID. No11 is a good responder to said anticancer treatment.

The “relative abundance” of an OTU is defined as a percentage of the number of sequences grouped into this OTU to the total number of sequences in a fecal sample. OTU should group at least two 16S rRNA gene sequences (singletons, i.e. OTU containing only one sequence, are not considered).

The determination of gut microbial composition and of the relative abundance of a given OTU can be determined with classical and appropriate method known by the person skilled in the art. In a particular embodiment, said determination is performed on total DNA extracted from human fecal or mucosal or tissue sample by 16S rRNA gene sequencing, such as Sanger, 454 pyrosequencing, MiSeq or HiSeq technologies. Determination can also be performed by quantitative PCR technology with specific probes targeting either the specific OTU sequence or its affiliated bacterial isolate and any sequences having at least 97%, preferably 98%, 99% or 100%, identity with the nucleic sequence of said OTU.

Enrichment or depletion is described as a comparison between both groups, responders and non responders, and thus highly depends on methodological issues such as the sequencing technologies, the size of the cohorts etc . . . Moreover, each OTU is represented at a different level in relative abundance, some OTU relative abundance ranging from 0.01 to 0.1% of total number of sequences, some other from 0.5% to 35% of the total number of sequences. Preferably, in the method of the invention, OTU relative abundance is assessed by applying a detection threshold of either 0.2%, or preferably 1%, of the total number of sequences (per sample).

The table I below illustrates mean, median, range, 1st quartile and 3rd quartile of relative abundance of each OTUs (SEQ. ID. No1 to SEQ. ID. No16) in each response Group (ie RESPONDERS vs. NON RESPONDERS to a CTLA4 treatment, see the experimental part below). Two criteria have been defined, one taking into account a threshold of relative abundance of the OTU (crit 2), and a second one taking into account detection level of each OTU (crit 1).

TABLE I

| | | RESPONDERS | | | | | | Non RESPONDERS | | | | | | | |
|---|---|---|---|---|---|---|---|---|---|---|---|---|---|---|---|
| SEQ | | | | 1st | 3rd | | | | | 1st | 3rd | | | | |
| ID | | | | Quar- | Quar- | | | | | Quar- | Quar- | | | RESPONSE | RESPONSE |
| N° | | Mean | MEDIAN | tile | tile | MIN | MAX | Mean | MEDIAN | tile | tile | MIN | MAX | (crit1) | (crit2) |
| 1 | denovo- | 0.878 | 0.636 | 0.282 | 1.459 | 0 | 2.87 | 0.076 | 0.000 | 0.000 | 0.012 | 0 | 1.0 | >0.28% | Detected |
| | 3066 | | | | | | | | | | | | | | at a 0.2% |
| | | | | | | | | | | | | | | | threshold |
| 2 | denovo- | 1.398 | 1.152 | 0.022 | 1.734 | 0 | 4.73 | 0.267 | 0.000 | 0.000 | 0.024 | 0 | 2.5 | >0.024% | Detected |
| | 991 | | | | | | | | | | | | | | at a 0.2% |
| | | | | | | | | | | | | | | | threshold |
| 3 | denovo- | 0.592 | 0.282 | 0.263 | 0.574 | 0 | 2.28 | 0.141 | 0.019 | 0.000 | 0.086 | 0 | 1.2 | >0.26% | Detected |
| | 4666 | | | | | | | | | | | | | | at a 0.2% |
| | | | | | | | | | | | | | | | threshold |
| 4 | denovo- | 0.685 | 0.177 | 0.130 | 0.804 | 0 | 2.92 | 0.027 | 0.000 | 0.000 | 0.000 | 0 | 0.3 | >0.13% | Detected |
| | 5905 | | | | | | | | | | | | | | at a 0.2% |
| | | | | | | | | | | | | | | | threshold |
| 5 | denovo- | 11.074 | 12.974 | 7.138 | 15.871 | 0 | 20.04 | 1.928 | 0.523 | 0.156 | 1.101 | 0 | 12.0 | >7.1% | Detected |
| | 3795 | | | | | | | | | | | | | | at a 1% |
| | | | | | | | | | | | | | | | threshold |
| 6 | denovo- | 2.710 | 1.905 | 1.493 | 3.224 | 0 | 9.76 | 0.831 | 0.020 | 0.000 | 0.167 | 0 | 7.5 | >1.49% | Detected |
| | 4787 | | | | | | | | | | | | | | at a 1% |
| | | | | | | | | | | | | | | | threshold |
| 7 | denovo- | 0.444 | 0.565 | 0.115 | 0.636 | 0 | 1.11 | 0.061 | 0.000 | 0.000 | 0.000 | 0 | 0.4 | >0.11% | Detected |
| | 453 | | | | | | | | | | | | | | at a 0.2% |
| | | | | | | | | | | | | | | | threshold |
| 8 | denovo- | 0.005 | 0.000 | 0.000 | 0.000 | 0 | 0.04 | 0.632 | 0.243 | 0.030 | 0.706 | 0 | 3.1 | <0.03% | Not |
| | 4054 | | | | | | | | | | | | | | Detected |
| | | | | | | | | | | | | | | | at a 0.2% |
| | | | | | | | | | | | | | | | threshold |
| 9 | denovo- | 0.346 | 0.000 | 0.000 | 0.000 | 0 | 2.98 | 2.502 | 1.668 | 0.013 | 4.340 | 0 | 8.0 | <0.013% | Not |
| | 3816 | | | | | | | | | | | | | | Detected |
| | | | | | | | | | | | | | | | at a 0.2% |
| | | | | | | | | | | | | | | | threshold |
| 10 | denovo- | 0.024 | 0.000 | 0.000 | 0.000 | 0 | 0.13 | 0.649 | 0.449 | 0.187 | 0.641 | 0 | 3.3 | <0.18% | Not |
| | 3657 | | | | | | | | | | | | | | Detected |
| | | | | | | | | | | | | | | | at a 0.2% |
| | | | | | | | | | | | | | | | threshold |
| 11 | denovo- | 0.070 | 0.000 | 0.000 | 0.000 | 0 | 0.61 | 0.619 | 0.180 | 0.030 | 0.422 | 0 | 3.9 | <0.03% | Not |
| | 5800 | | | | | | | | | | | | | | Detected |
| | | | | | | | | | | | | | | | at a 0.2% |
| | | | | | | | | | | | | | | | threshold |
| 12 | denovo- | 2.558 | 2.138 | 0.384 | 2.935 | 0 | 7.60 | 0.921 | 0.039 | 0.000 | 0.072 | 0 | 8.2 | >0.3% | Detected |
| | 5178 | | | | | | | | | | | | | | at a 0.2% |
| | | | | | | | | | | | | | | | threshold |
| 13 | denovo- | 0.038 | 0.000 | 0.000 | 0.000 | 0 | 0.24 | 0.627 | 0.380 | 0.000 | 0.898 | 0 | 2.6 | <0.24% | Not |
| | 3073 | | | | | | | | | | | | | | Detected |
| | | | | | | | | | | | | | | | at a 0.2% |
| | | | | | | | | | | | | | | | threshold |
| 14 | denovo- | 0.638 | 0.000 | 0.000 | 0.000 | 0 | 4.08 | 8.591 | 7.929 | 0.180 | 13.004 | 0 | 35.0 | <0.18% | Not |
| | 3428 | | | | | | | | | | | | | | Detected |
| | | | | | | | | | | | | | | | at a 0.2% |
| | | | | | | | | | | | | | | | threshold |
| 15 | denovo- | 0.092 | 0.000 | 0.000 | 0.000 | 0 | 0.44 | 3.139 | 0.957 | 0.000 | 6.437 | 0 | 14.3 | <0.4% | Not |
| | 892 | | | | | | | | | | | | | | Detected |
| | | | | | | | | | | | | | | | at a 0.2% |
| | | | | | | | | | | | | | | | threshold |
| 16 | denovo- | 0.708 | 0.565 | 0.099 | 1.131 | 0 | 1.95 | 0.275 | 0.000 | 0.000 | 0.019 | 0 | 2.1 | >0.02% | Detected |
| | 2582 | | | | | | | | | | | | | | at a 0.2% |
| | | | | | | | | | | | | | | | threshold |

According to another embodiment, the present invention relates to a method for predicting the response of an individual to an anticancer treatment with anti-CTL4 molecules comprising the steps of:

(i) determining gut microbial OTU in a fecal sample of said individual;

(ii) determining the presence or the absence of at least one OTU comprising a nucleotide fragment of sequence having at least 97%, preferably 98%, 99% or 100%, identity with a nucleic sequence selected in the group consisting of: denovo3066 of SEQ. ID. No1, denovo991 of SEQ. ID. No2, denovo4666 of SEQ. ID. No3, denovo5905 of SEQ. ID. No4, denovo3795 of SEQ. ID. No5, denovo4787 of SEQ. ID. No6, denovo453 of SEQ. ID. No7, denovo4054 of SEQ. ID. No8, denovo3816 of SEQ. ID. No9, denovo3657 of SEQ. ID. No10, denovo5800 of SEQ. ID. No11 and denovo5178 of SEQ. ID. No12, by applying a detection threshold of 0,2% of the total number of sequences (per sample) to define an OTU's presence or absence, or applying quantitative PCR detection with specific OTU or bacterial isolate targeting probes; wherein individual having a gut microbiote showing the presence of at least one OTU selected in the group consisting of denovo3066 of SEQ. ID. No1, denovo991 of SEQ. ID. No2, denovo4666 of SEQ. ID. No3, denovo5905 of SEQ. ID. No4, denovo3795 of SEQ. ID. No5, denovo4787 of SEQ. ID. No6, denovo453 of SEQ. ID. No7 and denovo5178 of SEQ. ID. No12 and/or showing the absence of at least one OTU selected in the group consisting of denovo4054 of SEQ. ID. No8, denovo3816 of SEQ. ID. No9, denovo3657 of SEQ. ID. No10 and denovo5800 of SEQ. ID. No11 is a good responder to said anticancer treatment.

A patient that is a “good responder to an anticancer treatment” is a patient who is affected with a cancer and who will show a clinically significant response after receiving said anticancer treatment; the clinically significant response may be assessed by clinical examination (body weight, general status, pain and palpable mass, if any), biomarkers and imaging studies (ultrasonography, CT scan, PAT scan, MRI).

As used herein, “cancer” means all types of cancers. In particular, the cancers can be solid or non solid cancers. Non limitative example of cancers are head and neck squamous cell carcinoma, Hodgkin's lymphoma, urothelial cancer of the bladder, mismatch repair deficient colorectal cancer, gastric cancer and merkel cell carcinoma.

The term “treatment” herein refers to any reduction of the progression, severity and/or duration of cancer.

According to a specific embodiment, the individual is a patient with metastatic melanoma.

According to another specific embodiment, anti-CTL4 molecule is an anti-CTL4 antibody, such as ipilimumab.

Accordingly, the method of the invention allows to determine whether a patient with metastatic melanoma is likely to benefit from a treatment with ipilimumab by analysis the gut microbiota in a feces sample from said patient.

Gut microbiote relates to the population of microorganisms living in the intestine of any organism belonging to the animal kingdom; in the present invention, said organism is preferably a human. The gut microbial composition evolves throughout the entire life and is the result of different environmental influences.

Dysbiosis refers to the deleterious loss of balance in gut microbial composition that may arise in some specific situations.

The fecal sample is collected at any moment before deciding the beginning of the anticancer treatment or at any moment during said treatment; preferably, the fecal sample is collected before the start of the anticancer treatment. In any case, it is collected in a patient without colitis.

OTU means operational taxonomic unit; in the present invention, an OTU regroups bacterial 16S sequences sharing the same or similar (at least 97% of sequence identity) and is named “denovo#”. Each OTU is affiliated to a bacterial species, either isolated or not, cultured or uncultured when sharing 98% or more sequence similarity with a sequence described in public databases.

In a particular embodiment, step (ii) of the method of the invention consists in determining the relative abundance or the presence or the absence of at least two, three, four, five or six sequences having at least 97%, preferably 98%, 99% or 100%, identity with the nucleic sequence of OTUs selected in the group consisting of: denovo3066 of SEQ. ID. No1, denovo991 of SEQ. ID. No2, denovo4666 of SEQ. ID. No3, denovo5905 of SEQ. ID. No4, denovo3795 of SEQ. ID. No5, denovo4787 of SEQ. ID. No6, denovo453 of SEQ. ID. No7, denovo4054 of SEQ. ID. No8, denovo3816 of SEQ. ID. No9, denovo3657 of SEQ. ID. No10, denovo5800 of SEQ. ID. No11, denovo5178 of SEQ. ID. No12, denovo3073 of SEQ. ID. No13, denovo3428 of SEQ. ID. No14, denovo892 of SEQ. ID. No15 and denovo2582 of SEQ. ID. No16.

Areas under the curves (AUC) have been calculated for several combinations of OTUs to be predictive to the response to anti CTLA4 treatment, the 12 combinations presented in the Table II below had an good predictive value with AUC >0.90:

TABLE II

| OTUs Combination (SEQ. ID. N^{o}) | AUC |
|---|---|
| 7 + 8 + 9 + 10 + 11 + 12 | 0.9 |
| 8 + 9 + 10 + 11 + 12 | 0.91 |
| 1 + 6 + 7 + 8 + 9 + 10 | 0.9215 |
| 1 + 7 + 8 + 9 + 10 | 0.9215 |
| 1 + 6 + 9 + 10 | 0.9084 |
| 1 + 7 + 9 + 10 | 0.9152 |
| 1 + 8 + 9 + 10 | 0.9215 |
| 1 + 9 + 10 | 0.9281 |
| 1 + 9 + 10 + 13 | 0.9215 |
| 1 + 9 + 10 + 14 | 0.9215 |
| 1 + 9 + 10 + 15 | 0.9019 |
| 1 + 9 + 10 + 16 | 0.9215 |

According to one specific embodiment of the method of the invention, step (ii) aims to determine relative abundance (enrichment or depletion) of the above listed OTUs combination; the following relative abundance of said combinations of OTUs in the gut microbiote of an individual means that said individual is a good responder to said anticancer treatment:

| Combination | enrichment | depletion |
|---|---|---|
| A | SEQ. ID. N^{o}: 7, 12 | SEQ. ID. N^{o}: 8, 9, 10, 11 |
| B | SEQ. ID. N^{o}: 12 | SEQ. ID. N^{o}: 8, 9, 10, 11 |
| C | SEQ. ID. N^{o}: 1, 6, 7 | SEQ. ID. N^{o}: 8, 9, 10 |
| D | SEQ. ID. N^{o}: 1, 7 | SEQ. ID. N^{o}: 8, 9, 10 |
| E | SEQ. ID. N^{o}: 1, 6 | SEQ. ID. N^{o}: 9, 10 |
| F | SEQ. ID. N^{o}: 1, 7 | SEQ. ID. N^{o}: 9, 10 |
| G | SEQ. ID. N^{o}: 1 | SEQ. ID. N^{o}: 8, 9, 10 |
| H | SEQ. ID. N^{o}: 1 | SEQ. ID. N^{o}: 9, 10 |
| I | SEQ. ID. N^{o}: 1 | SEQ. ID. N^{o}: 9, 10, 13 |
| J | SEQ. ID. N^{o}: 1 | SEQ. ID. N^{o}: 9, 10, 14 |
| K | SEQ. ID. N^{o}: 1 | SEQ. ID. N^{o}: 9, 10, 15 |
| L | SEQ. ID. N^{o}: 1, 16 | SEQ. ID. N^{o}: 9, 10 |

According to another specific embodiment of the method of the invention, step (ii) aims to determine the presence of the absence of the above listed OTUs combination; the following results observed for said combinations of OTUs in the gut microbiote of an individual means that said individual is a good responder to said anticancer treatment:

| Combination | presence | absence |
|---|---|---|
| A | SEQ. ID. N^{o}: 7, 12 | SEQ. ID. N^{o}: 8, 9, 10, 11 |
| B | SEQ. ID. N^{o}: 12 | SEQ. ID. N^{o}: 8, 9, 10, 11 |
| C | SEQ. ID. N^{o}: 1, 6, 7 | SEQ. ID. N^{o}: 8, 9, 10 |
| D | SEQ. ID. N^{o}: 1, 7 | SEQ. ID. N^{o}: 8, 9, 10 |
| E | SEQ. ID. N^{o}: 1, 6 | SEQ. ID. N^{o}: 9, 10 |
| F | SEQ. ID. N^{o}: 1, 7 | SEQ. ID. N^{o}: 9, 10 |
| G | SEQ. ID. N^{o}: 1 | SEQ. ID. N^{o}: 8, 9, 10 |
| H | SEQ. ID. N^{o}: 1 | SEQ. ID. N^{o}: 9, 10 |
| I | SEQ. ID. N^{o}: 1 | SEQ. ID. N^{o}: 9, 10, 13 |
| J | SEQ. ID. N^{o}: 1 | SEQ. ID. N^{o}: 9, 10, 14 |
| K | SEQ. ID. N^{o}: 1 | SEQ. ID. N^{o}: 9, 10, 15 |
| L | SEQ. ID. N^{o}: 1, 16 | SEQ. ID. N^{o}: 9, 10 |

The Table III below shows the correspondence between OTU and their affiliated bacterial isolate/species:

TABLE III

| | | | Corresponding |
|---|---|---|---|
| | | | bacterial 16S |
| | SEQ. ID. | | sequence |
| OTUs | N^{o} | Affiliated bacterial isolate/species | SEQ. ID. N^{o} |
| denovo3066 | 1 | Parabacteroides distasonis | 17 |
| denovo991 | 2 | uncultured bacterium NA48 AY975552 | 18 |
| | | (Clostridium XIVa/Fusicatenibacter | |
| | | saccharivorans) | |
| denovo4666 | 3 | butyrate producing bacterium SS2 1 | 19 |
| | | AY305319 (Anaerostipes hadrus) | |
| denovo5905 | 4 | uncultured bacterium C3 13 GQ896957 | 20 |
| | | (Ruminococcus/Oscillibacter valericigenes) | |
| denovo3795 | 5 | Faecalibacterium prausnitzii | 21 |
| denovo4787 | 6 | Gemmiger formicilis | 22 |
| denovo453 | 7 | uncultured bacterium RL246 aai74e12 | 23 |
| | | DQ793623 (Unclassified Clostridiales/ | |
| | | Faecalibacterium sp. canine oral taxon 147) | |
| denovo4054 | 8 | Bacteroides ovatus | 24 |
| denovo3816 | 9 | Faecalibacterium prausnitzii | 25 |
| denovo3657 | 10 | Clostridium sp. XB90/Eisenbergiella | 26 |
| | | massiliensis | |
| denovo5800 | 11 | uncultured bacterium REC1M 21 AY343160 | 27 |
| | | (Oscillibacter sp. Marseille-P 2778) | |
| denovo5178 | 12 | butyrate producing bacterium L2 21 | 28 |
| | | AJ270477/Eubacterium rectale | |
| denovo3073 | 13 | Bacteroides uniformis JCM5828 3 EU136680 | 29 |
| denovo3428 | 14 | Bacteroides sp ALA Bac AM117579 | 30 |
| denovo892 | 15 | Bacteroides uniformis JCM 5828T AB050110 | 31 |
| denovo2582 | 16 | Blautia obeum 1 33 AY 169419/Blautia | 32 |
| | | wexlerae JCM17041 | |

According to a further embodiment, step (ii) of the method of the invention consists in detecting the relative abundance and/or the presence or absence of at least one of the bacterial isolate/species affiliated to the OTU of SEQ. ID. No1 to 12 by culturing a feces sample on appropriate culture medium and in appropriate culture conditions,

wherein enrichment or presence in an individual's gut microbiote of bacterial isolate/species selected in the group consisting of Parabacteroides distasonis, uncultured bacterium NA48 AY975552 (Clostridium XIVa/Fusicatenibacter saccharivorans), butyrate producing bacterium SS2 1 AY 305319 (Anaerostipes hadrus), uncultured bacterium C3 13 GQ896957 (Ruminococcus/Oscillibacter valericigenes), Faecalibacterium prausnitzii, Gemmiger formicilis, uncultured bacterium RL246 aai74e12 DQ793623 (Unclassified Clostridiales/Faecalibacterium sp. canine oral taxon 147) and butyrate producing bacterium L2 21 AJ270477/Eubacterium rectale and/or the depletion or absence in said individual's gut microbiote of bacterial isolate/species selected in the group consisting of Bacteroides ovatus, Faecalibacterium prausnitzii, Clostridium sp. XB90/Eisenbergiella massiliensis and uncultured bacterium REC1M 21 AY343160 (Oscillibacter sp. Marseille P2778) means that said individual is a good responder to said anticancer treatment.

According to another embodiment, step (ii) of the method according to the present invention consists in determining the relative abundance of at least one OTU comprising a nucleic acid fragment of 300 to 1500 pb having a sequence of at least 97%, preferably 98%, 99% or 100%, identity with a nucleic acid fragment sequence of a 16S rRNA selected in the group consisting of: SEQ. ID. No16, SEQ. ID. No17, SEQ. ID. No18, SEQ. ID. No19, SEQ. ID. No20, SEQ. ID. No21, SEQ. ID. No22, SEQ. ID. No23, SEQ. ID. No24, SEQ. ID. No25, SEQ. ID. No26, SEQ. ID. No27 and SEQ. ID. No28.

From the OTUs population described in the overall cohort, metabolomic profiles have been deduced in silico applying the PICRUSt algorithm combined with LEFSe.

A specific set of metabolic pathways (described as KEGG IDs) have been highlighted specifically associated with response to anti CTLA4 or toxicity to anti CTLA4 (see experimental part IV).

To further validate this in silico obtained results and/or highlight new metabolites, both known or unknown, that would discriminate patients for their response to antiCTLA4, metabolomic profiling of the 26 patients' feces at baseline has been evaluated and allowed the identification of sets of metabolites associated with response to anti CTLA4.

The present invention thus also relates to a method for predicting the response of an individual to an anticancer treatment with anti-CTL4 molecules comprising the step of determining the presence or the absence of at least one metabolite (described as KEGG ID) selected in the group consisting of:

Membrane and intracellular structural molecules

Other glycan degradation

Lipopolysaccharide biosynthesis

Other ion coupled transporters

Aminosugar and nucleotidesugar metabolism

Carbon fixation pathways in prokaryotes

Purine metabolism

Galactose metabolism

Sphingolipid metabolism

Chaperones and folding catalysts

Fructose and mannose metabolism

Lysosome

Oxidative phosphorylation

Alanine_aspartate and glutamate metabolism

Peptidases

Secretion system

Porphyrin and chlorophyll metabolism

Two_component system

Flagellar assembly

Bacterial chemotaxis

Bacterial motility proteins

ABC transporters

Transporters

in a biological sample of said individual, wherein the presence of at least one metabolite selected in the group consisting of:

Secretion system

Porphyrin and chlorophyll metabolism

Two_component system

Flagellar assembly

Bacterial chemotaxis

Bacterial motility proteins

ABC transporters

Transporters

is associated with a good response to said anti-cancer treatment and/or the presence of at least one metabolite selected in the group consisting of:

Membrane and intracellular structural molecules

Other glycan degradation

Lipopolysaccharide biosynthesis

Other ion_coupled transporters

Aminosugar and nucleotidesugar metabolism

Carbon fixation pathways in prokaryotes

Purine metabolism

Galactose metabolism

Sphingolipid metabolism

Chaperones and folding catalysts

Fructose and mannose metabolism

Lysosome

Oxidative phosphorylation

Alanine aspartate and glutamate metabolism

Peptidases

is associated with a poor response to said anti-cancer treatment.

The present invention thus also relates to a method for predicting the response of an individual to an anticancer treatment with anti-CTL4 molecules comprising the step of determining the presence or the absence of the following metabolites (first set of metabolites):

Trehalose-Sucrose-Isomaltose

Guanine

D-Raffinose

3,4-Dihydroxyphenylacetic acid

12-Hydroxydodecanoic acid

in a biological sample of said individual, wherein the presence of the following metabolites:

Trehalose-Sucrose-Isomaltose

Guanine

D-Raffinose

3,4-Dihydroxyphenylacetic acid

is associated with a good response to said anti-cancer treatment and/or the presence of at least one metabolite selected in the group consisting of:

12-Hydroxydodecanoic acid

is associated with a poor response to said anti-cancer treatment. The present invention further relates to a method for predicting the response of an individual to an anticancer treatment with anti-CTL4 molecules comprising the step of determining the presence or the absence of the following metabolites (second set of metabolites):

1-Methylxanthine

Pipecolinic acid

2-Isopropylmalic acid

in a biological sample of said individual, wherein the presence of the following metabolites:

2-Isopropylmalic acid

is associated with a good response to said anti-cancer treatment and/or the presence of at least one metabolite selected in the group consisting of:

1-Methylxanthine

Pipecolinic acid

is associated with a poor response to said anti-cancer treatment.

Optionally, the method making use of the second set of metabolites may be combined with the method making use of the first set of metabolites.

The present invention further relates to a method for predicting the response of an individual to an anticancer treatment with anti-CTL4 molecules comprising the step of determining the presence or the absence of the following metabolites (third set of metabolites) in combination with the first and the second set of metabolites:

Alpha D Amino adipic acid

Kynurenic acid

Methylimidazoleacetic acid

3,4 dihydroxyhydrocinnamic acid

Cotinine

2 Methylnicotinamide

in a biological sample of said individual, wherein the presence of the following metabolites:

Kynurenic acid

is associated with a good response to said anti-cancer treatment and/or the presence of at least one metabolite selected in the group consisting of:

Alpha D Amino adipic acid

Methylimidazoleacetic acid

3,4 dihydroxyhydrocinnamic acid

Cotinine

2 Methylnicotinamide

is associated with a poor response to said anti-cancer treatment.

The present invention also relates to:

a composition comprising one or more purified bacterial strains of species selected from the group consisting of Lachnospiraceae butyrate producing bacterium, Bacteroides ovatus, Ruminococcaceae clostridiales bacterium, Blautia obeum, Fusicatenibacter saccharivorans, Roseburia inulinivorans, Gemmiger formicilis, and Faecalibacterium prausnitzii; in other embodiments, said composition comprises two or more, three or more, four or more, five or more, six or more, seven or more, eight or more, nine or more, or ten or more bacterial strains;

a composition comprising one or more purified bacterial strains with 16S rRNA sequences having at least 97% sequence identity with bacterial strains of species selected from the group consisting of Lachnospiraceae butyrate producing bacterium, Bacteroides ovatus, Ruminococcaceae clostridiales bacterium, Blautia obeum, Fusicatenibacter saccharivorans, Roseburia inulinivorans, Gemmiger and Faecalibacterium prausnitzii; in other embodiments, said purified bacterial strains have 16S rRNA sequences having at least 97%, at least 98%, or at least 99% sequence identity and/or said composition comprises two or more, three or more, four or more, five or more, six or more, seven or more, eight or more, nine or more, or ten or more bacterial strains;

a composition comprising one or more purified bacterial strains selected from the group consisting of butyrate producing bacterium SS2-1, Bacteroides ovatus CIP 103756, Clostridiales bacterium CIEAF 026, Blautia obeum 1-33, Fusicatenibacter saccharivorans TT-111, Roseburia inulinivorans type strain A2-194, Gemmiger formicilis ATCC 27749 X2-56, butyrate producing bacterium L2-21 and Faecalibacterium prausnitzii L2-6; in other embodiments, said composition comprises two or more, three or more, four or more, five or more, six or more, seven or more, eight or more, nine or more, or ten or more bacterial strains;

a composition comprising one or more purified bacterial strains with 16S rRNA sequences having at least 97% sequence identity with bacterial strains selected from the group consisting of butyrate producing bacterium SS2-1, Bacteroides ovatus CIP 103756, Clostridiales bacterium CIEAF 026, Blautia obeum 1-33, Fusicatenibacter saccharivorans TT-111, Roseburia inulinivorans type strain A2-194, Gemmiger formicilis ATCC 27749 X2-56, butyrate producing bacterium L2-21 and Faecalibacterium prausnitzii L2-6; in other embodiments, said purified bacterial strains have 16S rRNA sequences having at least 97%, at least 98%, or at least 99% sequence identity and/or said composition comprises two or more, three or more, four or more, five or more, six or more, seven or more, eight or more, nine or more, or ten or more bacterial strains;

a composition comprising one or more purified bacterial strains of species selected from the group consisting of Lachnospiraceae butyrate producing bacterium, Gemmiger formicilis, and Faecalibacterium prausnitzii; in other embodiments, said composition comprises two or more, three or more, four or more, or five or more bacterial strains;

a composition comprising one or more purified bacterial strains with 16S rRNA sequences having at least 97% sequence identity with bacterial strains of species selected from the group consisting of Lachnospiraceae butyrate producing bacterium, Gemmiger formicilis, and Faecalibacterium prausnitzii; in other embodiments, said purified bacterial strains have at least 97%, at least 98%, or at least 99% sequence identity and/or said composition comprises two or more, three or more, four or more, or five or more bacterial strains;

a composition comprising one or more purified bacterial strains selected from the group consisting of Gemmiger formicilis ATCC 27749 X2-56, butyrate producing bacterium L2-21 and Faecalibacterium prausnitzii L2-6; in other embodiments, said composition comprises two or more, three or more, four or more, or five or more bacterial strains;

a composition comprising one or more purified bacterial strains with 16S rRNA sequences having at least 97% sequence identity with bacterial strains selected from the group consisting of Gemmiger formicilis ATCC 27749 X2-56, butyrate producing bacterium L2-21 and Faecalibacterium prausnitzii L2-6; in other embodiments, said purified bacterial strains have at least 97%, at least 98%, or at least 99% sequence identity and/or said composition comprises two or more, three or more, four or more, or five or more bacterial strains.

According to specific embodiments, each of the previous cited compositions may be such that:

at least 10%, at least 20%, at least 30%, at least 40%, at least 50%, at least 60%, at least 70%, at least 80%, at least 90%, or at least 100% of the bacterial strains belong to the Firmicutes phylum;

less than 100%, less than 90%, less than 80%, less than 70%, less than 60%, less than 50%, less than 40%, less than 30%, less than 20%, or less than 10%, of the bacterial strains belong to the genus Bacteroides;

it does not include bacterial strains of the genus Bacteroides;

it does not include bacterial strains of the species Bacteroides fragilis or Bacteroides thetaiotamicron;

it does not include bacterial strain Faecalibacterium prausnitzii A2-165;

said bacterial strains are lyophilized;

it further comprises an immune checkpoint inhibitor; in a specific embodiment, the immune checkpoint inhibitor is a PD-1 inhibitor, PD-L1 inhibitor, or CTLA-4 inhibitor; when the immune checkpoint inhibitor is a CTLA-4 inhibitor, said CTLA-4 inhibitor may be ipilimumab or tremelimumab, preferably, said CTLA-4 inhibitor is ipilimumab; when the immune checkpoint inhibitor is a inhibitor is a PD-1 inhibitor, said PD-1 inhibitor may be nivolumab or pembrolizumab; when the immune checkpoint inhibitor is a PD-L1 inhibitor, said PD-L1 inhibitor may be atezolizumab, avelumab or durvalumab.

The present invention further relates to a pharmaceutical composition comprising anyone of the compositions of the invention and a pharmaceutically acceptable carrier.

In an embodiment, said pharmaceutical composition is formulated for delivery to the intestine.

In an embodiment, said pharmaceutical composition is in the form of a capsule.

In an embodiment, said pharmaceutical composition is formulated for oral administration.

In an embodiment, said pharmaceutical composition comprises a pH sensitive composition comprising one or more enteric polymers.

The present invention also relates to a method of treating cancer comprising administering a pharmaceutically effective amount of the pharmaceutical composition of the invention to treat the cancer in the subject. In an embodiment, said cancer is melanoma. In an embodiment, said method further comprises a step of determining if the subject has developed colitis.

In some embodiments, the bacterial strains are purified. Any of the bacterial strains described herein may be isolated and/or purified, for example, from a source such as a culture or a microbiota sample (e.g., fecal matter). The bacterial strains used in the compositions provided herein generally are isolated from the microbiome of healthy individuals. As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

In some embodiments, the composition further comprises an immune checkpoint inhibitor (also referred to as “checkpoint inhibitor”). Immune checkpoints are regulatory pathways within the immune system that are involved in maintaining immune homeostasis (e.g., self-tolerance, modulating the duration and extent of an immune response) to minimize cellular damage due to aberrant immune responses. Inhibitors of immune checkpoints, herein referred to as “immune checkpoint inhibitors,” specifically inhibit immune checkpoints and may have a stimulatory or inhibitory effect on the immune response. Without wishing to be bound by any particular theory, it is thought in art that different cancers and tumors may manipulate immune checkpoints to evade detection and/or modulate the immune response.

In some embodiments, the immune checkpoint inhibitor is a PD-1 inhibitor, PD-L1 inhibitor, or CTLA-4 inhibitor. (See e.g., Vesely M D, Annu Rev

Immunol 2011, 29:235-271; Nature Reviews Cancer 12, 252-264). In some embodiments of the compositions provided herein, the immune checkpoint inhibitor is a PD-1 inhibitor, PD-L-1 inhibitor, CTLA-4 inhibitor, IDO1 inhibitor, LAG3 inhibitor or TIM3 inhibitor.

In some embodiments, the immune checkpoint inhibitor is a CTLA-4 inhibitor. In some embodiments, the CTLA-4 inhibitor is an anti-CTLA-4 antibody. Examples of anti-CTLA-4 antibodies include, without limitation, ipilimumab, tremelimumab (CP-675,206), 9H10, 4F10, and 9D9. In some embodiments, the CTLA-4 inhibitor is ipilimumab. In some embodiments, the CTLA-4 inhibitor is tremelimumab.

In some embodiments, the immune checkpoint inhibitor is a PD-1 inhibitor. In some embodiments, the PD-1 inhibitor is nivolumab. In some embodiments, the PD-1 inhibitor is pembrolizumab.

In some embodiments, the immune checkpoint inhibitor is a PD-L1 inhibitor. In some embodiments, the PD-L1 inhibitor is atezolizumab. In some embodiments, the PD-L1 inhibitor is avelumab. In some embodiments, the PD-L1 inhibitor is durvalumab.

It should further be appreciated that multiple immune checkpoint inhibitors may be used in the methods and compositions disclosed herein. For instance, in a non-limiting example, the methods described herein include the administration of both a PD-1 inhibitor and a CTLA-4 inhibitor.

Any of the compositions described herein may be administered to a subject in a pharmaceutically effective amount (also referred to herein as “therapeutically effective amount”) or a dose of a therapeutically effective amount to treat the cancer (e.g., melanoma or metastatic melanoma). The terms “treat” or “treatment” refer to reducing or alleviating one or more of the symptoms associated with cancer.

Any of the compositions described herein, including the pharmaceutical compositions comprising the compositions, may contain bacterial strains in any form, for example in an aqueous form, such as a solution or a suspension, embedded in a semi-solid form, in a powdered form or freeze-dried form. In some embodiments, the composition or the bacterial strains of the composition are lyophilized. In some embodiments, a subset of the bacterial strains in a composition is lyophilized. Methods of lyophilizing compositions, specifically compositions comprising bacteria, are well known in the art. See, e.g., U.S. Pat. Nos. 3,261,761; 4,205,132; PCT Publications WO 2014/029578 and WO 2012/098358, herein incorporated by reference in their entirety. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may be subsequently be combined with a pharmaceutical excipient. In some embodiments, the bacterial strain is a lyophilized cake. In some embodiments, the compositions comprising the one or more bacterial strains are a lyophilized cake.

The bacterial strains of the composition can be manufactured using fermentation techniques well known in the art. In some embodiments, the active ingredients are manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the composition of bacterial strains may be formulated for administration as a pharmaceutical composition. The term “pharmaceutical composition” as used herein means a product that results from the mixing or combining of at least one active ingredient, such as any two or more purified bacterial strains described herein, and one or more inactive ingredients, which may include one or more pharmaceutically acceptable excipient (also referred to herein as “pharmaceutically acceptable carrier”). An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions of the disclosure can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like.

In some embodiments, the compositions are formulated for delivery to the intestines (e.g., the small intestine and/or the colon). In some embodiments, the compositions are formulated with an enteric coating that increases the survival of the bacteria through the harsh environment in the stomach. The enteric coating is one which resists the action of gastric juices in the stomach so that the bacteria which are incorporated therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). (See e.g., Zhang, AAPS PharmSciTech, (2016) 17 (1), 56-67).

The bacteria may also be formulated for rectal delivery to the intestine (e.g., the colon). Thus, in some embodiments, the bacterial compositions may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH sensitive compositions, more specifically, buffered sachet formulations or enteric polymers that release their contents when the pH becomes alkaline after the enteric polymers pass through the stomach. When a pH sensitive composition is used for formulating the pharmaceutical preparation, the pH sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon and rectum), has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon and rectum) by providing formulations with the appropriate pH sensitivity. (See e.g., Villena et al., Int J P harm 2015, 487 (1-2): 314-9).

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

The compositions comprising bacterial strains are formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition is formulated such that the bacteria of the composition, or a portion thereof, remain viable after passage through the stomach of the subject. In some embodiments, the pharmaceutical composition is formulated for rectal administration., e.g. as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

Dosages of the active ingredients in the pharmaceutical compositions can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions of the present disclosure employed, the route of administration, the time of administration, the duration of the treatment, other drugs, compounds and/or materials used in combination with the particular compositions employed, the age, sex, weight, condition, general health and prior medical history of the subject being treated, and like factors. A physician, veterinarian or other trained practitioner, can start doses of the pharmaceutical composition at levels lower than that required to achieve the desired therapeutic effect and gradually increase the dosage until the desired effect is achieved. In general, effective doses of the compositions of the present disclosure, for the prophylactic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of multiple doses of any of the compositions described herein. In some embodiments, the composition is administered orally the subject once, twice, 3 times, 4 times, 5 times, 6 times, 7 times, 8 times, 9 times, or at least 10 times.

Aspects of the present disclosure include methods and compositions for the treatment of cancer in a subject. In some embodiments, the subject has cancer or is at risk of developing cancer. Examples of cancers that can be treated according to the methods provided herein, include without limitation, carcinoma, glioma, mesothelioma, melanoma (e.g., metastatic melanoma), lymphoma, leukemia, adenocarcinoma, breast cancer, ovarian cancer, cervical cancer, glioblastoma, multiple myeloma, prostate cancer, Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease, AIDS-associated primary effusion lymphoma, neuroectodermal tumors, or rhabdomyosarcoma. In some embodiments of the methods provided herein, the cancer is prostate cancer, bladder cancer, non-small cell lung cancer, urothelial carcinoma, melanoma, Merkel cell cancer, or renal cell carcinoma. In some embodiments, the cancer is melanoma, non-small cell lung cancer (NSCLC), Hodgkin's lymphoma, head and neck cancer, renal cell cancer, bladder cancer, or Merkel cell carcinoma.

In some embodiments, the cancer is melanoma and the anticancer therapy involves administering a CTLA-4 inhibitor (e.g., ipilimumab, tremelimumab) and one or more of the bacterial compositions provided herein. In some embodiments, the cancer is melanoma, NSCLC, Hodgkin's lymphoma, renal cancer, head and neck cancer and the anticancer therapy involves administering a PD-1 inhibitor (e.g., pembrolizumab, nivolumab) and one or more of the bacterial compositions provided herein. In some embodiments, the cancer is bladder cancer, NSCLC, or Merkel cell carcinoma and the anticancer therapy involves administering a PD-L1 inhibitor (e.g., atezolizumab, avelumab, durvalumab) and one or more of the bacterial compositions provided herein.

### FIGURES LEGENDS

FIG. 1. Gut microbiota at baseline and during colitis in patients with metastatic melanoma treated with ipilimumab.

A. Principal component analysis representation of patient distribution based on bacterial genera composition between baseline visit (V₁) and colitis. Seven patients had fecal samples collected at both baseline and time of colitis onset. Monte-Carlo simulated p-value=0.0059. Component 1 explains 13.59% of variance; component 2 explains 11.45% of variance.

B. Relative abundance of dominant (>1% of total reads) gut microbial genera significantly reduced during colitis (Vₜₒₓ) as compared to baseline (V₁). LACHN: Lachnospiracea incertae sedis, RUM: Ruminococcus, BLAU: Blautia, CL_IV: Clostridium IV, EUB: Eubacterium, Unc_LACH: unclassified Lachnospiraceae, PSEUD: Pseudoflavonifractor (paired t-test p<0.05 for all genera).

C. Proportions as percent of total reads of significantly impacted bacterial isolates during colitis (Vₜₒₓ) as compared to baseline (V₁). Only significant (paired t-test p<0.05) data are presented.

FIG. 2. Baseline gut microbiota as a predictor of response to ipilimumab

A. Principal component analysis representation of patients' distribution based on bacterial genera composition at baseline (V₁) depending on the benefit of ipilimumab treatment; LT Benefit: long-term benefit (in green) vs Poor Benefit (in dark red). Component 1 explains 12.86% of variance; component 2 explains 8.67% of variance. Monte-Carlo simulated p-value=0.00899.

B. Boxplot of the percentages of 4 dominant (>1% of total reads) genera differentially represented between both groups, i.e. Bacteroides, Faecalibacterium, Clostridium XIVa and Gemmiger ; LT_Benefit: long-term benefit vs Poor Benefit; *:p<0.05; **:p<0.001.

C. Inter-class principal component analysis representation of patient distribution based on bacterial genera composition at baseline (V₁) depending on overall survival time following ipilimumab treatment; GS0_6: overall survival ranging from 0 to 6 months (n=2), GS6_9: overall survival ranging from 6 to 9 months (n=5), GS9_12: overall survival ranging from 9 to 12 months (n=4), GS12_18: overall survival ranging from 12 to 18 months (n=7), GSsup18: overall survival superior to 18 months (n=8). Monte-Carlo simulated p-value=0.01098.

D. Percentages of 3 specific OTUs highlighted as biomarkers at baseline (V₁) of overall survival duration greater than 18 months. Each patient's microbiota are presented in the graph. GS0_6: overall survival ranging from 0 to 6 months (n=2), GS6_9: overall survival ranging from 6 to 9 months (n=5), GS9_12: overall survival ranging from 9 to 12 months (n=4), GS12_18: overall survival ranging from 12 to 18 months (n=7), GSsup18: overall survival greater than 18 months (n=8).

FIG. 3. Baseline Gut microbiota composition predicts clinical response to ipilimumab.

A. Inter-class principal component analysis representation of patient's stratification into 3 statistically robust clusters (A, B and C) at baseline (Vᵢ). Monte-Carlo simulated p-value=0.00099.

B. Bacterial genera discriminating the 3 different clusters at baseline were assessed with a Random Forest analysis, and confirmed with a Wilcoxon test. Percentage of reads for each of these 6 genera is represented for each cluster.

C. Baseline gut microbiota composition and overall survival (OS). Kaplan-Meier survival curves of patients classified into two groups according to clusters, Cluster A versus Cluster B.

D. Baseline gut microbiota composition and progression free survival. Kaplan-Meier curves of patients classified into two groups according to clusters, Cluster A versus Cluster B. P values are indicated on each graph.

FIG. 4. Gut microbiota composition at baseline predicts ipilimumab-induced colitis.

A. Boxplot of relative abundance of the 2 dominant phyla Firmicutes and Bacteroidetes at baseline between patients prone to or resistant to ipilimumab-induced colitis. A Wilcoxon test has been applied to assess significance and p-values are indicated on the graph.

B. Colitis cumulative incidence (Gray's test) of patients classified into two groups according to clusters, Cluster A versus Cluster B. P values are indicated on the graph.

C. OTUs predictive of colitis development during ipilimumab treatment. LEfSe uses Linear Discriminant Analysis (LDA) to estimate the effect size of each differentially abundant feature (i.e. bacterial OTU). On the left panel, OTUs in dark grey are biomarkers of colitis development (Colitis). OTUs light grey are biomarkers of the absence of colitis development (No_Colitis). Best biomarkers show the highest absolute LDA score with a minimal threshold of 3.5 (i.e. OTU denovo592 for the No_Colitis group and OTU denovo3795 for the Colitis group). Right panel indicates the taxonomic affiliation of each of these OTUs. Sim.: similarity between the OTU read and the first assigned isolate 16S sequence, assessed by the RDP Sab_score. The RDP S_ab score is a percentage of shared 7-mers between two sequences.

FIG. 5. Systemic immune status and microbiota composition

A-C. Baseline (V₁) percentages and absolute numbers of fresh whole blood CD4⁺ T cells, Treg cells (% CD4⁺CD3⁺CD25⁺Foxp3⁺ within CD4⁺CD3⁺ cells) ; α4⁺β7⁺ among CD4⁺ T and CD8⁺ T cells, serum concentrations of IL-6, IL-8 and sCD25 were analyzed and compared between patients belonging to Cluster A and patients belonging to Cluster B (A); long term clinical benefit (LT benefit) and poor clinical benefit (Poor benefit) (B); patients with colitis (Colitis) and patients without colitis (No colitis) (C). Each dot represents one patient. P values are indicated on each graph; ns means “not significant”; Mann Whitney tests were used.

(D) The expression of ICOS on CD4+21 T cells was monitored in fresh whole blood before ipilimumab treatment (V₁) and after one or two injections of ipilimumab (V₂-3). Serum concentration of sCD25 was monitored prior to ipilimumab treatment (V₁) and after one or two injections of ipilimumab (V₂-3). Percentage of conventional CD4+ 24 T cells (Tconv) was defined as % CD3+CD4+ T excluding Treg cells. Graphs depict specific changes in immune activation over the course of ipilimumab treatment in patients belonging to Cluster A (white circles) and patients belonging to Cluster B (black circles). Each dot represents one patient. 1 P values are indicated on each graph. Mann Whitney (A-C) and Wilcoxon matched-pairs signed rank (D) tests were used.

FIG. 6. Graphical representation of individual repartition of 5 metabolites in fecal samples of patients.

FIG. 7. Schematic representation of the ROC curve was built based on these 14 metabolites and the area under the curve showed good prediction value (AUC=0.8602941).

FIG. 8. This figure shows that while the combination of the 1st set of metabolites (5 selected biomarkers) show good predictive value (AUC>0.8), addition of the Sets #2 and #3 increase stratification power with an AUC>0.85, ameliorating hence the biomarkers specificity.

FIG. 9. Graphical representation of the repartition of the 14 metabolites within the 2 groups of patients in the boxplots (y-axis=peak area).

FIG. 10. Graph showing that a specific set of metabolic pathways (described as KEGG IDs) have been highlighted specifically associated with toxicity to anti CTLA4.

FIG. 11. Graph showing that a specific set of metabolic pathways (described as KEGG IDs) have been highlighted specifically associated with response to anti CTLA4.

FIG. 12. Graph showing the main metabolites present in feces that help discriminate between Responders and Non Responders to aCTLA4 treatment.

### EXAMPLES

### I. Patients, Materials and Methods

Patients

Patients with MM treated with ipilimumab were prospectively enrolled at the Gustave Roussy Cancer Campus between March 2013 and December 2014. Patients were informed of the study and consented to participate. This study was approved by the Kremlin Bicetre Hospital Ethics Committee (GOLD study: SC12-018; ID-RCB-2012-A01496-37) and all procedures were performed in accordance with the Declaration of Helsinki. Patients had a pre-specified clinical workup; feces and blood were collected at baseline (V₁), prior to each ipilimumab infusion (V₂, V₃, V₄), at the end of treatment (ie, 3 weeks after the last infusion; V₅) and, if present, at the time of colitis occurrence (VTox). Ipilimumab was administered intravenously every 3 weeks at a dose of 3 or 10 mg/Kg and could be continued after V₄, at a maintenance dose of one infusion every 12 weeks, in patients whose disease was controlled (response or stable disease). When immune-mediated enterocolitis (≥grade III) was suspected, patients were referred to the Gastroenterology Department of Bicetre Hospital. The diagnosis of ipilimumab-induced colitis was made in patients who had endoscopic signs of inflammation and no other cause of colitis, such as ischemia and infection (stool tests for bacterial pathogens and Clostridium difficile toxin had to be negative).

Response to ipilimumab was assessed by several criteria. Long-term clinical benefit was defined by response decrease of tumour burden≥50% relative to baseline, according to immune related response criteria [15, 16]) or stable disease (decrease of tumour burden of less than 50% but with less than 25% increase relative to nadir) for more than 6 months. Patients with poor benefit were defined as patients with a lack of long term benefit, i.e. with progression-free survival of less than 6 months (with immune related progressive disease defined as a confirmed increase in tumor burden ≥25% relative to nadir) [15, 16]. All responses were confirmed by a subsequent assessment no less than 4 weeks from the date first documented.

Bacterial Composition Assessment by High-Throughput Sequencing

Fecal samples were collected anaerobically at baseline (V₁), prior to each ipilimumab infusion (V₂, V₃, V₄), at the end of the treatment (V₅) and at the time of colitis (V_{Tox}) and were kept at −80° C. until analysis. Total DNA was extracted from fecal sample aliquots (˜150mg), as previously described using both physical and chemical lysis [17]. Culture-independent 16S rRNA gene sequencing was performed on 83 fecal samples (n=26 patients; Table S3). Both 454 pyrosequencing (Life Sciences, a Roche company, Branford, Conn., USA) and MiSeq (Illumina Inc, San Diego, Calif., USA) technologies were applied on the V₃-V_{4 16}S rRNA gene region. Gut bacterial composition was determined prospectively without knowledge of colitis or of clinical benefit, which was determined at the end of the study.

More precisely, for both pyrosequencing (454) and MiSeq sequencing, the V₃-V₄ region of the 16S rRNA gene was amplified with the following primers: V₃F <<TACGGRAGGCAGCAG>> (V₃F bac339F modified with R instead of G) (Wilson K H, et al. J Clin Microbiol. 1990) and V₄R <<GGACTACCAGGGTATCTAAT>>bac806R. For 454, applied quality filters were minimum length=250 bp, maximum length=600 bp, minimum quality threshold=25, maximum number of homopolymers=6. For MiSeq, the filters were minimum length=200 bp and minimum quality threshold=20. Applying these filters, an average of 1639 and 7377 reads/samples were kept for further analysis respectively with 454 and MiSeq technology. High quality reads were pooled, checked for chimeras, and grouped into Operational Taxonomic Units (OTUs) based on a 97% similarity threshold with uclust software from QIIME. Finally 462,312 high-quality reads (average 4,623 reads/sample) were analyzed and grouped into an average of 349 Operational Taxonomic Units (OTUs) per sample (97% similarity threshold; OTUs or phylotypes) based on uclust software from QIIME [1]. Estimates of phylotypes richness and diversity were calculated using both Shannon and Simpson indices on the rarefied OTU table (n=1,000 reads). Singletons were removed and phylogenetic affiliation of each OTU was done by using ribosomal database project taxonomy [2] and performed from phylum to species level.

The applied sequencing technology is indicated for each sample (n=83) in the Table S3, as is the number of samples used for comparisons within the different part of the study. Statistical analyses were applied to decipher the influence of ipilimumab treatment, colitis development, and other clinical factors on the microbiota and to highlight a combination of microbial groups and immunological defects significantly involved in colitis development in MM patients.

Statistical testing for significant differences between all combinations of two groups was conducted using either paired Student or the Mann-Whitney test. The statistical 1 language R was used for data visualization and to perform abundance-based principal component analysis (PCA) and inter-class PCA associated with Monte-Carlo rank testing on the bacterial genera.

The Random Forest classifier was applied to relative abundance data of distributed bacterial genera and OTUs to assess the variable importance of microbial components in the classification of each patient to a certain phenotype [3].

The random forest machine learning analyses were applied to identify bacterial genera level that differentiates fecal community composition between groups. The purpose of a classifier such as random forest is to learn a function that maps a set of input values or predictors (here, relative to genera abundances) to a discrete output value (here, relative to clinical groups).

Random forest is a powerful classifier that can use nonlinear relationships and complex dependencies between taxa. The degree of the success of the method is its ability to classify unseen samples correctly, estimated by training it on a subset of samples, and using it to categorize the remaining samples [3]. The cross-validation error is compared with the baseline error that would be achieved by always predicting the most common category. Random forest analysis was performed for each comparison on 5000 rarefied versions of the data.

Additionally, random forest assigns an importance score to each genus by estimating the increase in error caused by eliminating that genus from the set of predictors. It also reports the Gini importance of a variable, which is computed as the sum of the Gini impurity decrease of every node in the forest for which that variable was used for splitting. Moreover, the LEfSe (linear discriminant analysis effect size) algorithm [4] was applied for high dimensional discovery of biomarkers that discriminate between patients that will have colitis but who may also respond (or not) to ipilimumab. The algorithm is freely available at http:huttenhower.sph.harvard.edu/galaxy/. Finally, patients were statistically stratified based on their dominant gut microbial composition (genus level). Clustering of patients was performed with the k-means clustering algorithm and Calinski-Harabasz index 1 to select the optimal number of clusters as previously described [5]. The k-means clustering algorithm implemented in R package was used. Interclass PCA of genera composition with clusters as instrumental variable was also assessed, based on a Monte Carlo test with 1000 replicates.

Lastly, random forest analysis was also performed to identify bacterial genera that differentiated fecal community composition between the three identified clusters.

Fresh Whole Blood Immune Monitoring and Soluble Immune Markers

Blood samples were collected at baseline (V₁), prior to each ipilimumab infusion (V₂, V₃, V₄), at the end of treatment (V₅) and at the time of colitis (V_{Tox}). Phenotyping was performed on fresh whole blood or on fresh peripheral blood mononuclear cells isolated by Ficoll density gradient and frozen for later analyses (see supplementary materials). All serum samples were stored at −80° C. until further analysis of soluble immune markers of inflammation (IL-6, IL-8, IP-10, MCP-1, TNFα, sCD25) and bacterial translocation (sCD14). Details of immune monitoring can be found in supplementary materials. We monitored memory T cells and ICOS induction on T cells because previous studies had demonstrated that both could be related to clinical responses [18]. Regulatory T cells play a crucial role in the maintenance of immune homeostasis in the gut and are supposed to be a major target for anti-CTLA-4 treatment [19]. The heterodimer α4β7 plays a crucial role in the intestinal homing of T cells; we monitored α4⁺β7⁺ T cells to gain some insight in the mechanism of ipilimumab-induced colitis. We monitored soluble immune markers of inflammation and bacterial translocation as a complement to microbiota composition. Indeed, microbiota composition affects gut barrier function, local as well as systemic immunity [20-25] [26]

Statistical Analyses

A formal sample-size calculation was not performed for this pioneering study. Associations between microbiota dominant profile and immunological parameters were assessed with the Spearman correlation coefficient and a two-sided Wilcoxon test. No adjustment for multiple comparisons was made because of the exploratory component of the analyses. Fisher's exact test was used to examine the association between microbiota clusters and immune parameters; microbiota clusters and colitis, as well as microbiota clusters and clinical responses.

Overall survival (OS) and progression-free survival (PFS) according to intestinal microbiota (Cluster A vs. Cluster B) and according to the number of conventional CD4³⁰ T cells (low number of conventional CD4 vs. high number of conventional CD4) were estimated using the Kaplan-Meier method and compared using the log-rank test.

When considering the occurrence of toxicity, progression and death lead to informative censoring, because when patients are censored at progression or death, their risk of toxicity could differ from that of patients who are not censored. Competing risk analysis is a method to deal with competing events, in order to avoid selection bias induced by informative censoring.

Therefore, toxicity cumulative incidence function (CIF) was estimated through a competing risk analysis in which toxicity was the event of interest, death or progression was the competing event and patients alive without progression or toxicity at their last follow-up were censored from the date of last follow-up. The toxicity cumulative incidence functions in the different gut microbiota clusters and for the different conventional CD4 levels were estimated using the SAS macro %CIF and compared using the stratified Gray's test [27, 28]. These analyses were performed using SAS software, Version 9.4 (SAS Institute Inc., Cary, N.C., USA).

II. Results

Clinical Characteristics of the Patients

Fifty-five patients were included in the study and followed-up for at least 6 months. Among them, 13 patients did not receive ipilimumab, while four patients received only a single infusion of ipilimumab and died soon thereafter due to their melanoma; they were therefore excluded from further analyses. Among the 38 remaining patients, 26 provided fecal samples at baseline (i.e. before the first dose of ipilimumab) for microbiota analysis. Nine of these 26 patients (34.6%) had a long-term clinical benefit and seven (26.9%) developed an immune-mediated colitis.

Microbiota Composition is not Modified by Ipilimumab Treatment, except at the Time of Colitis

The gut microbiota was not significantly modified over the course of the ipilimumab treatment. Main bacterial phyla, Firmicutes and Bacteroidetes, remained stable over time. Even though bacterial genera proportions differed between the different time points, they were not significantly altered by ipilimumab treatment (Wilcoxon test p>0.05). Likewise, diversity was not altered by ipilimumab injections as assessed by both Shannon and Simpson indices. Altogether these data indicate that ipilimumab does not induce a gut microbial dysbiosis in patients with MM.

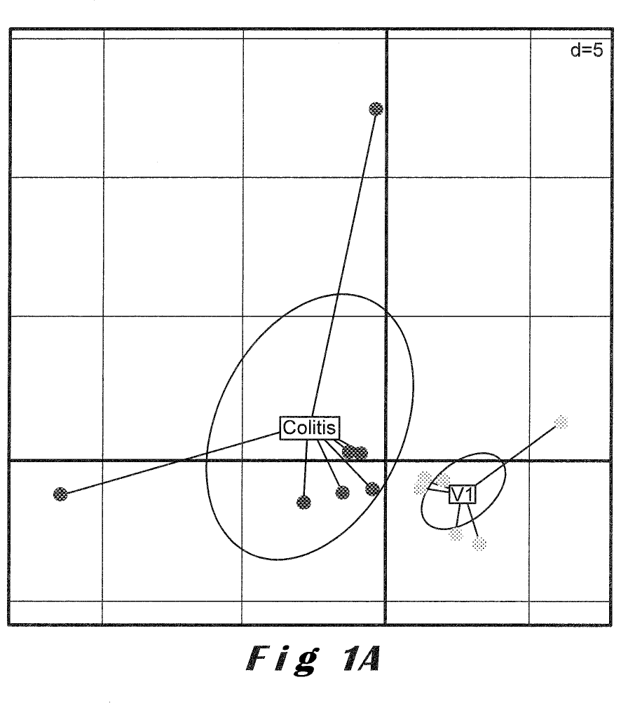

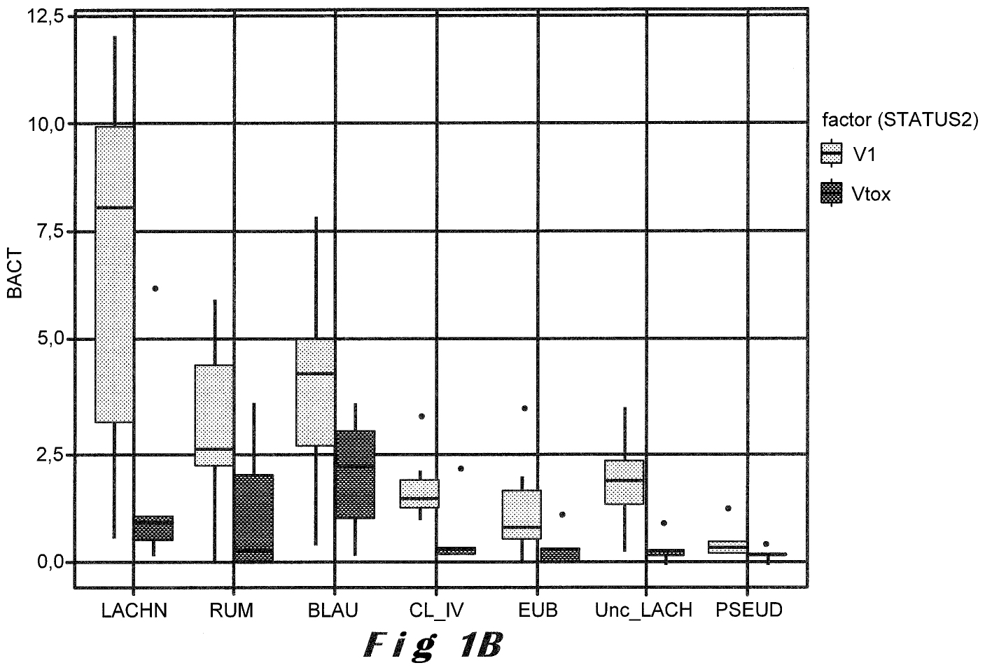

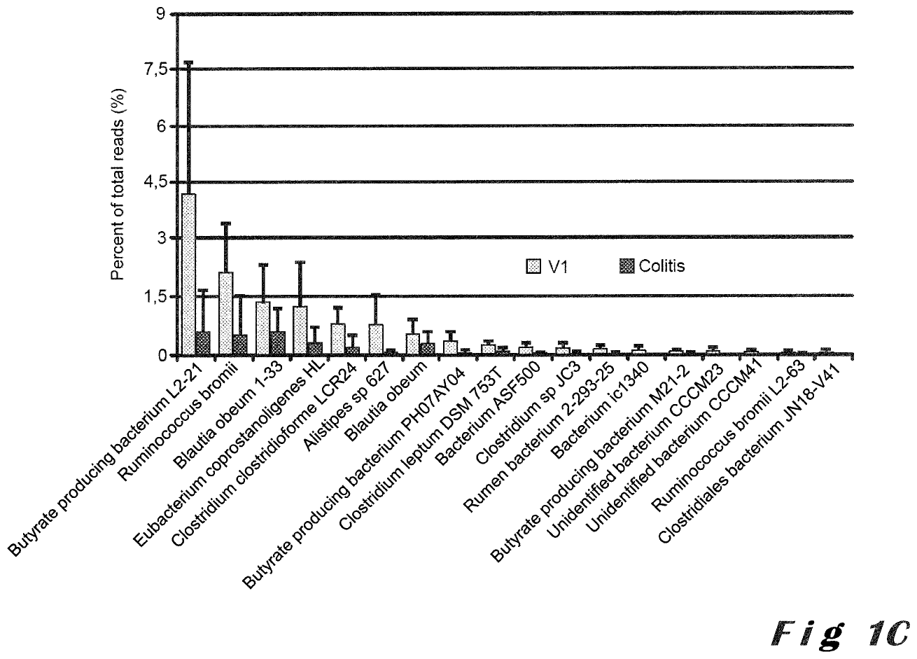

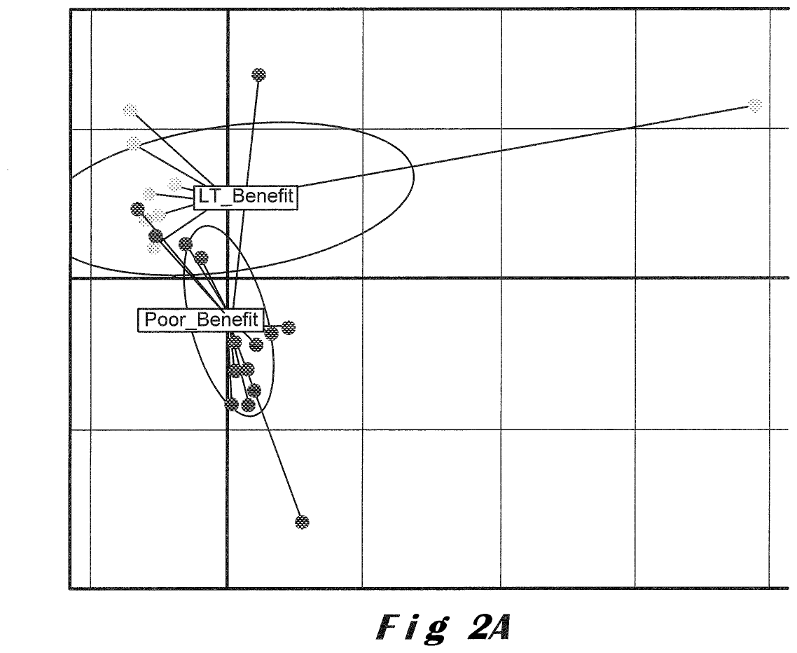

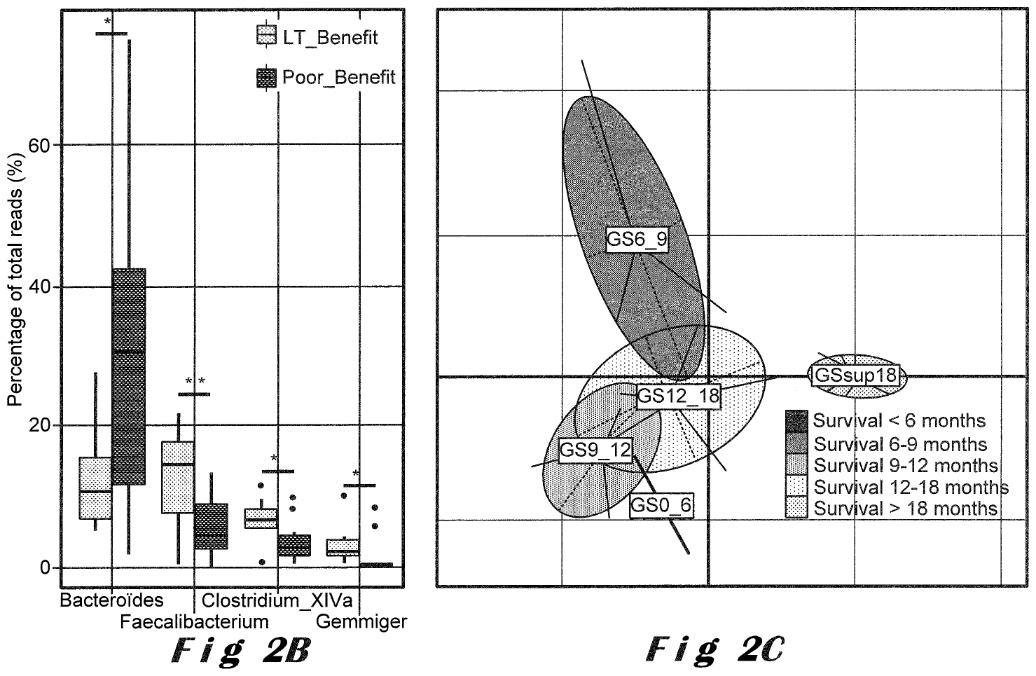

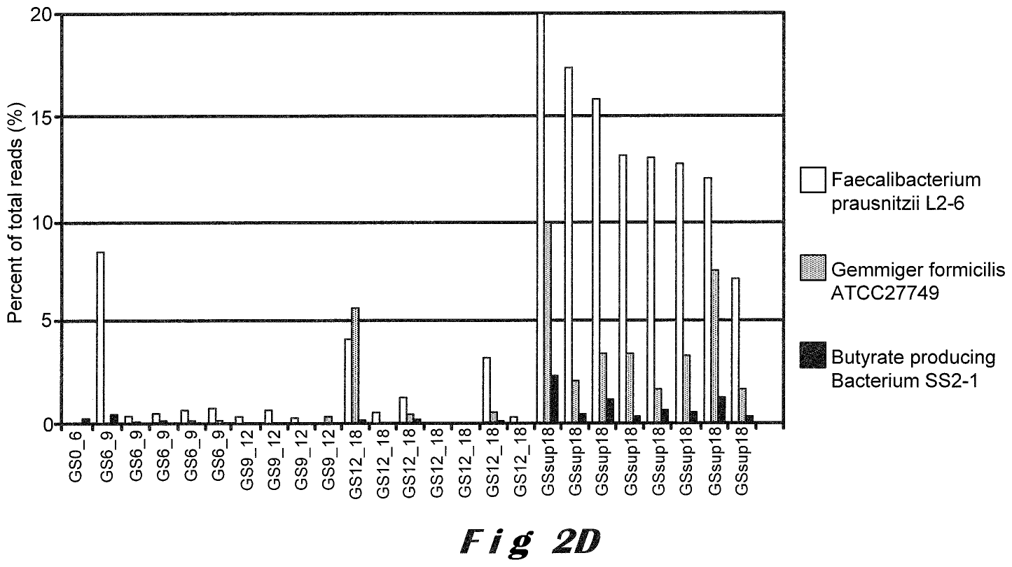

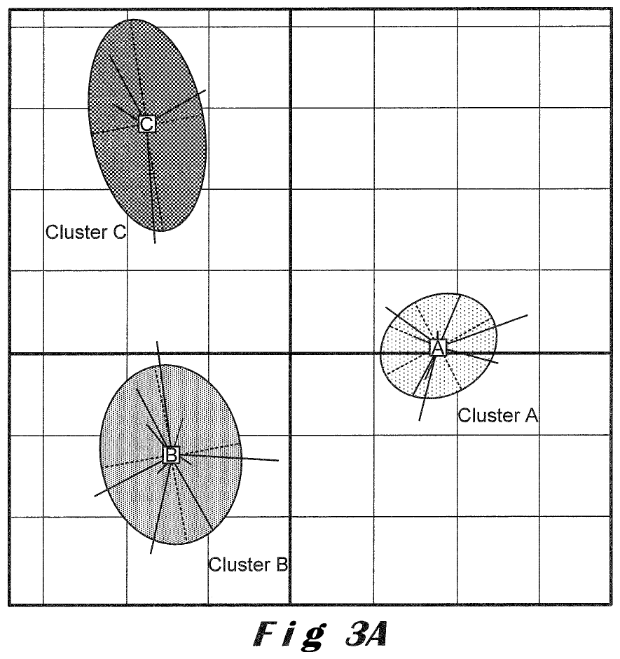

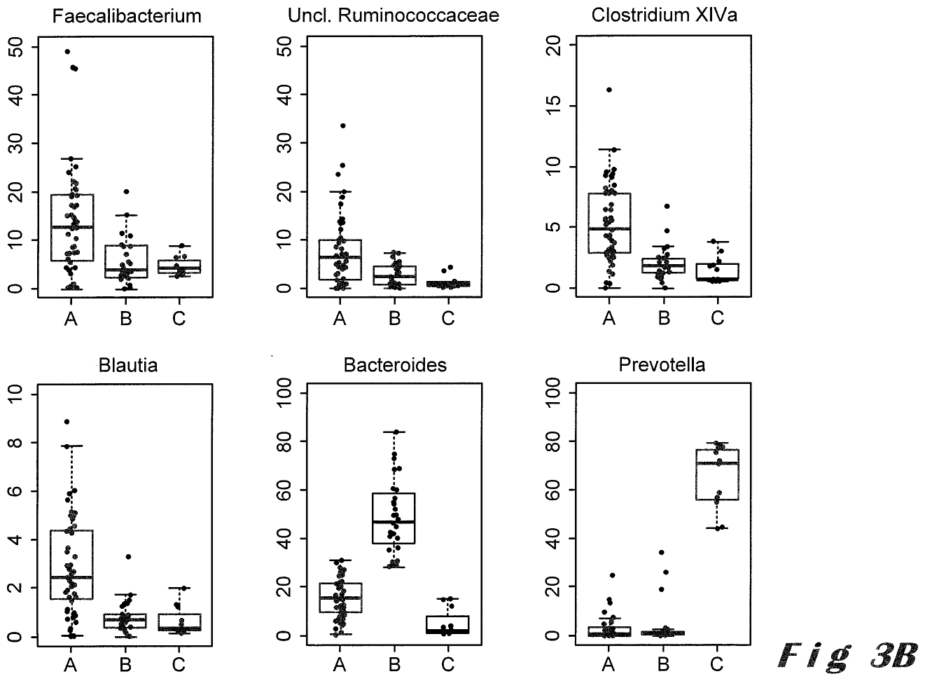

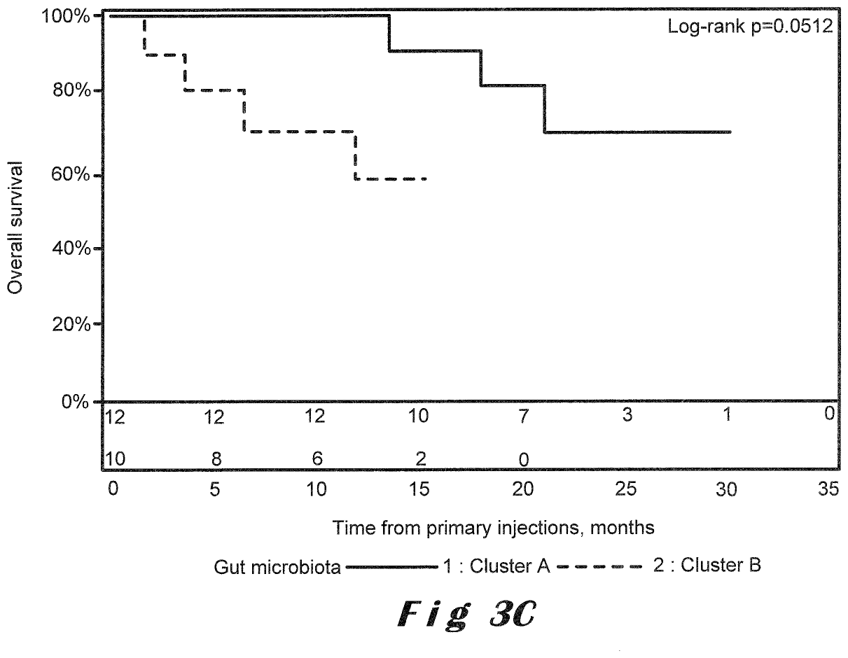

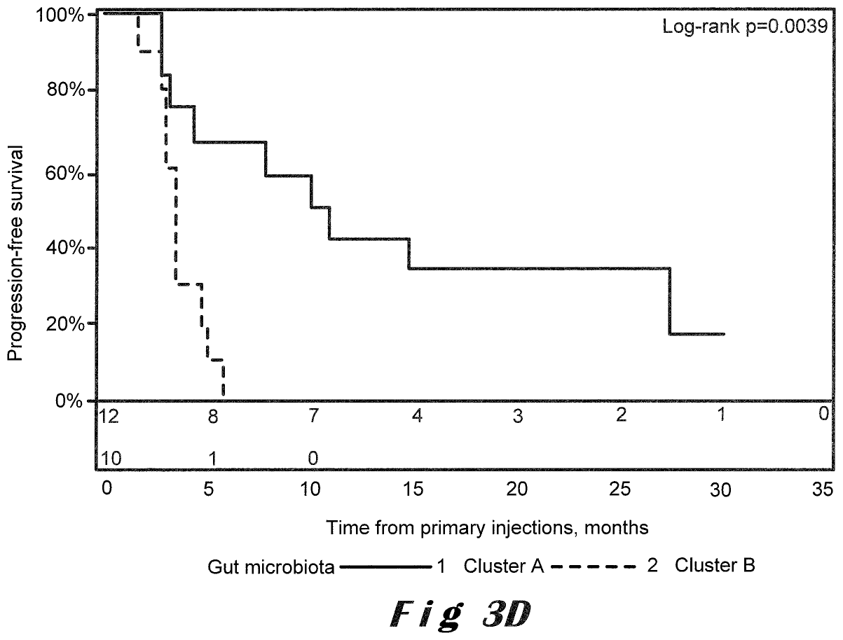

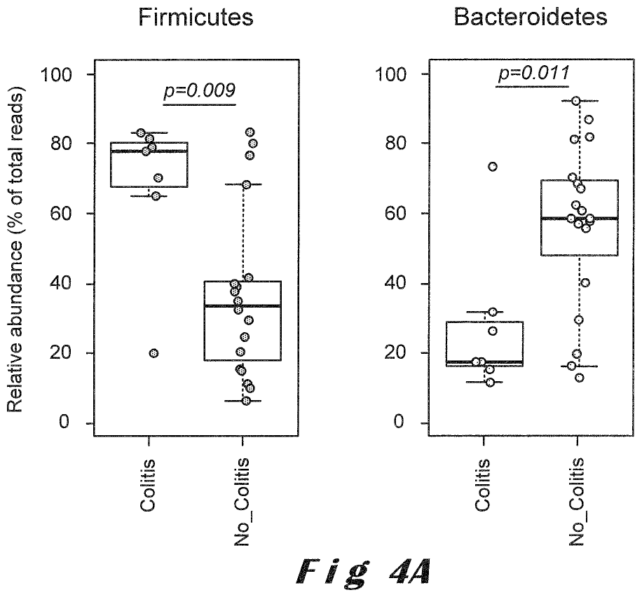

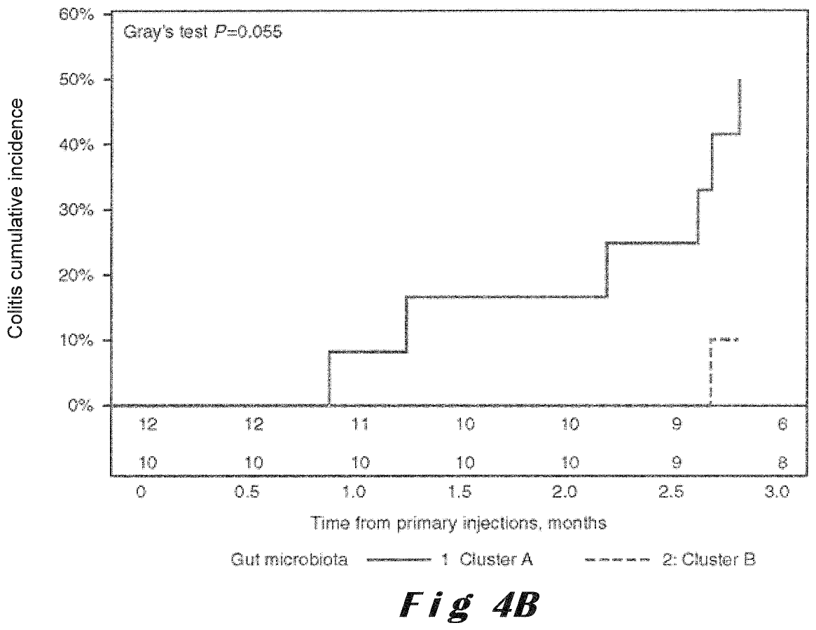

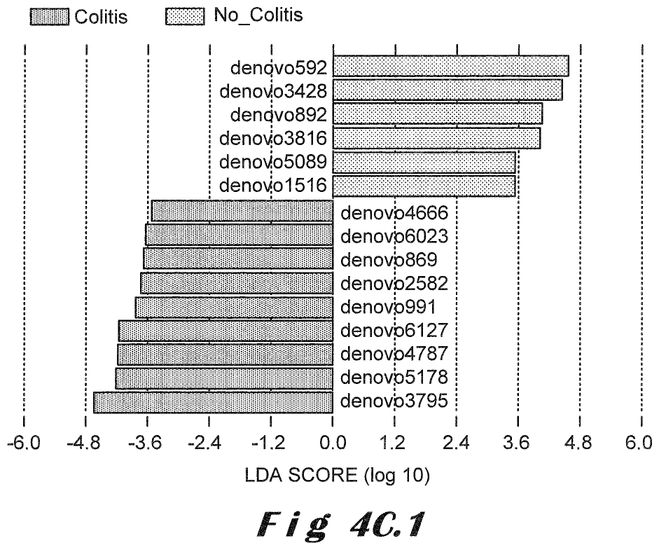

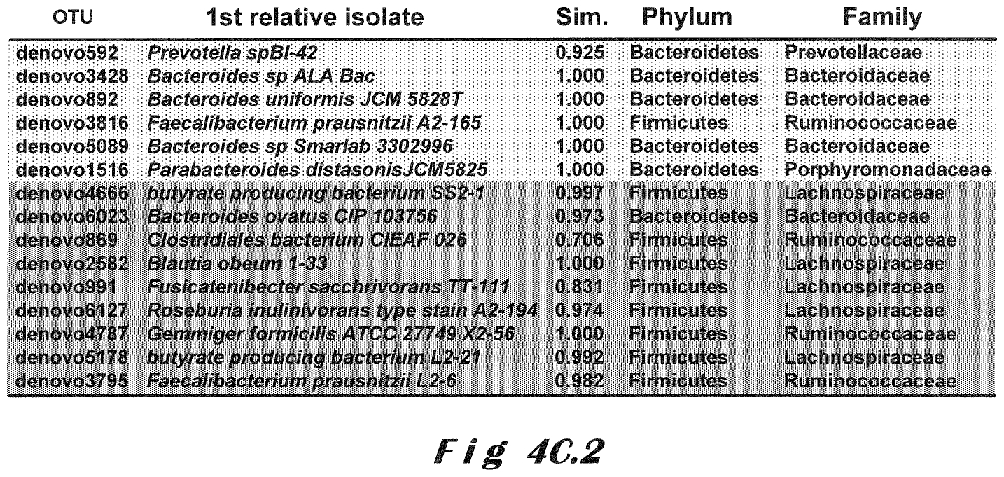

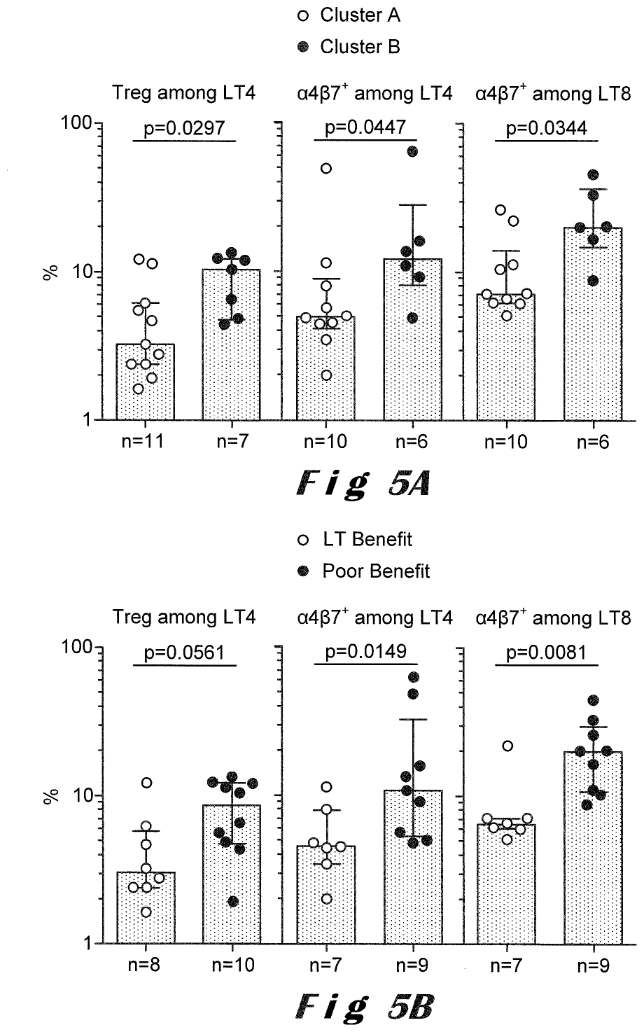

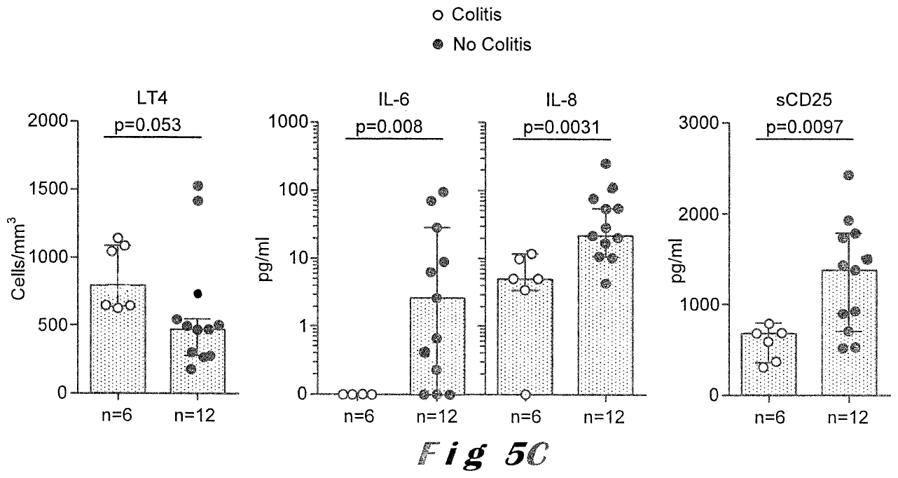

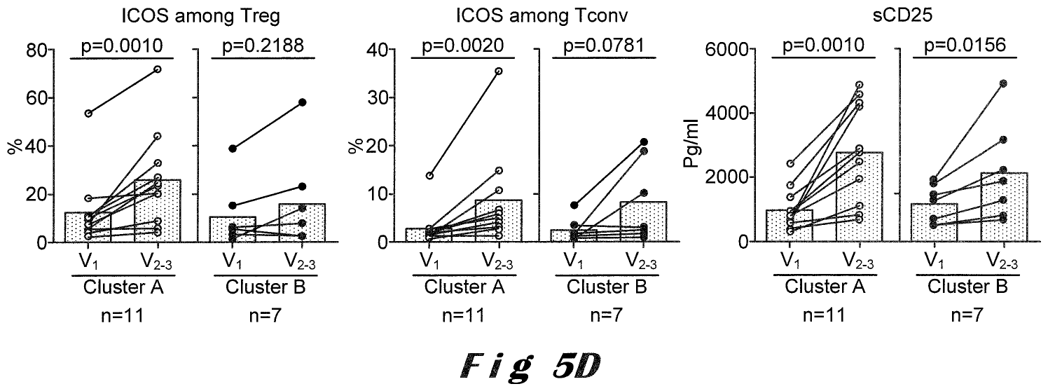

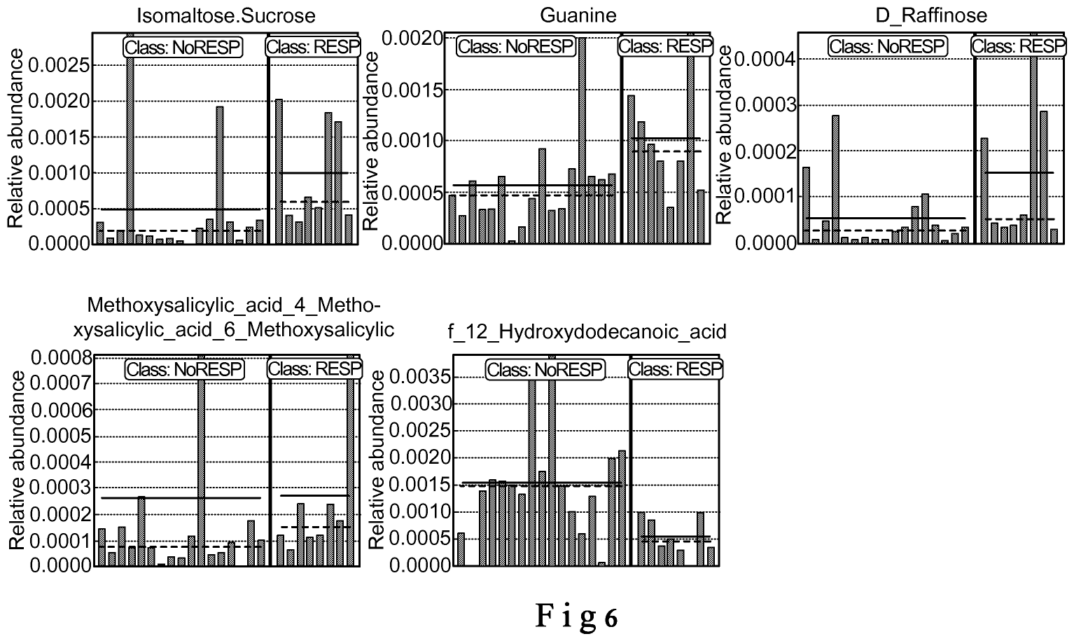

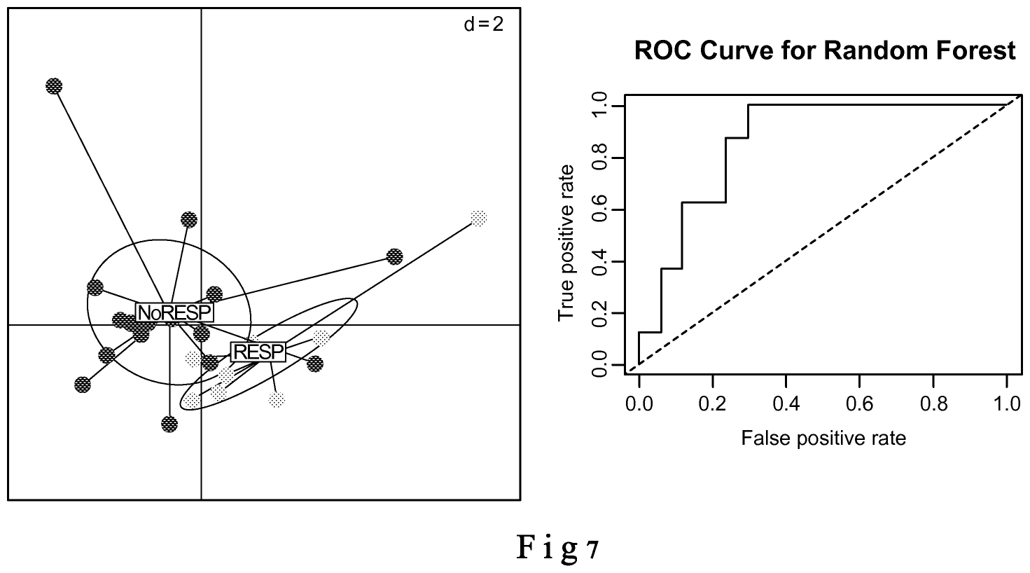

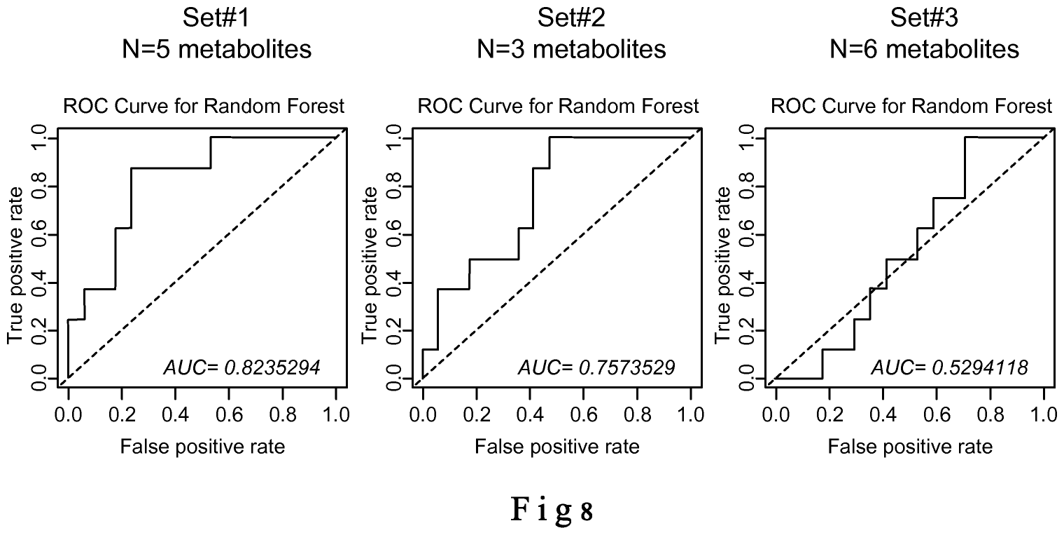

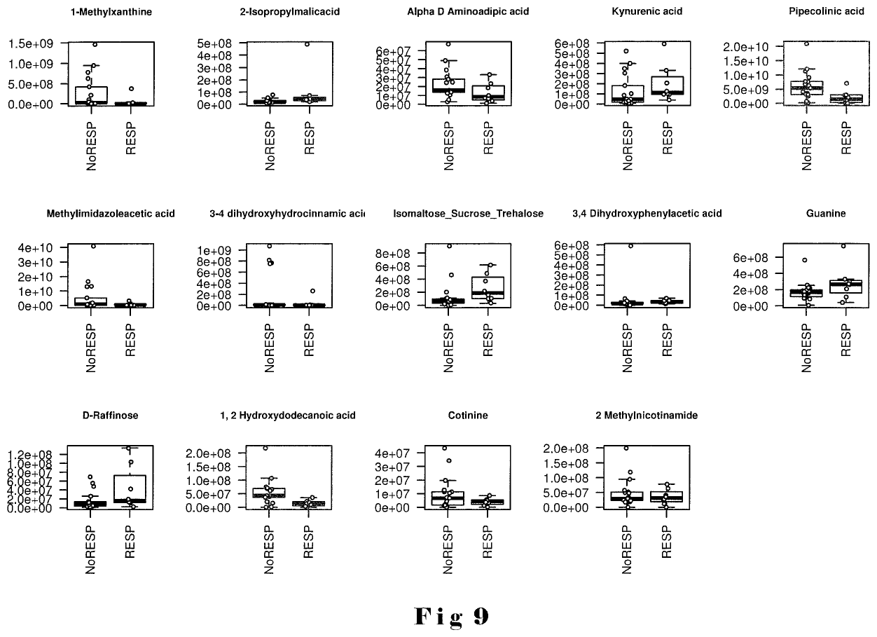

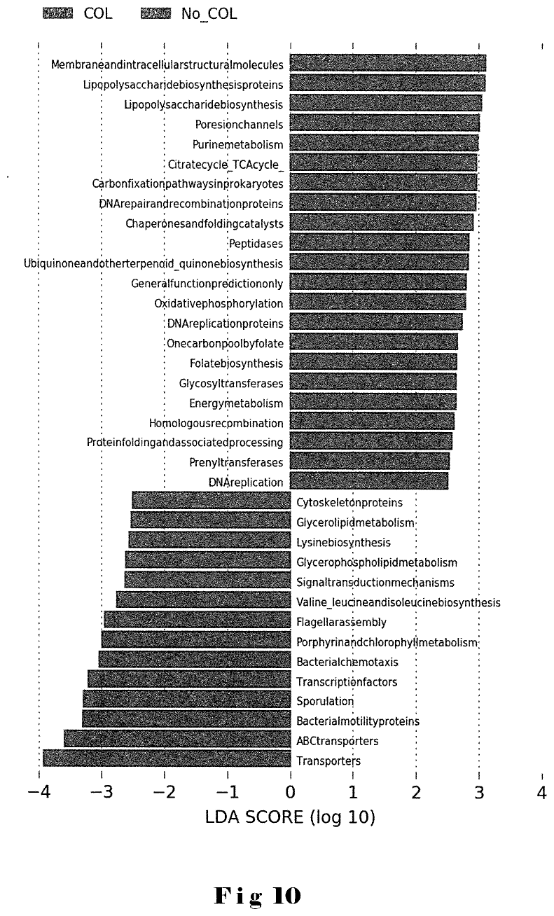

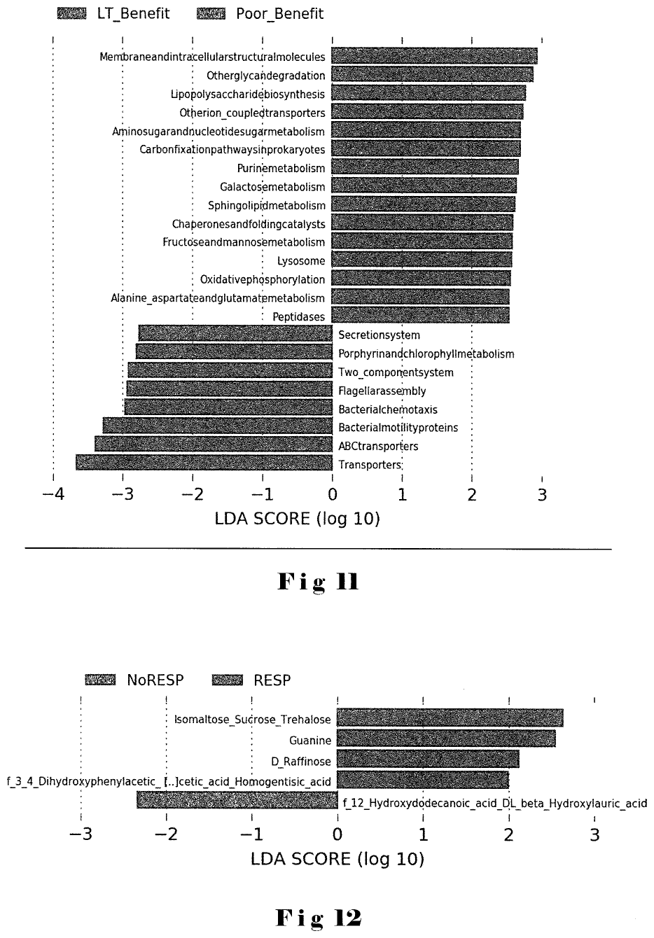

Seven patients provided fecal samples both at baseline (V₁) and during colitis (Vtox). Immune-related colitis was associated with a shift in the gut microbial composition as highlighted on the principal component analysis (PCA; FIG. 1A). These differences were significant at the family level (Wilcoxon test, p=0.0049) and at the genus level (p=0.0059). The proportions of seven genera were significantly reduced by at least 2 fold at Vₜₒₓ, compared to V₁ (FIG. 1B). They all belonged to the Firmicutes phylum (Ruminococcus, Lachnospiracea incertae sedis, Blautia, Clostridium IV, Eubacterium, unclassified Lachnospiraceae and Pseudoflavonifractor) and were dominant members of the microbiota. Similarly, 18 bacterial isolates, mostly Firmicutes, were significantly reduced between V₁ and Vₜₒₓ (paired Student t-test p<0.05, FIG. 1C). Finally, colitis was associated with a decreased bacterial diversity, as reflected by a lower Shannon diversity index at the time of colitis (V_{1 shannon}=5.58, V_{tox shannon}=4.65; paired student t-test p=0.042).

Baseline Microbiota Composition is Associated with Subsequent Clinical Response and Immune-Related Colitis

At V₁, inter-class PCA based on genera composition showed a significant clustering of patients depending on their clinical benefit in response to ipilimumab, either long-term clinical benefit or poor clinical benefit (Monte-Carlo test, p=0.00899; FIG. 2A). This was also true at finer taxonomic levels such as species or OTUs composition (p=0.00499 and p=0.00799 respectively). Moreover, fecal microbiota at V₁ allowed the correct classification of 84.6% of patients into either the long-term or poor clinical benefit category, and the accuracy reached more than 99% (5,000 bootstraps) for patients with poor benefit, as assessed by a Random Forest analysis. The receiver operating characteristic (ROC) curve provided an area under the curve (AUC) of 0.77, which indicates a fair classification based on overall microbial composition. Main genera accounting for this stratification are Faecalibacterium, Gemmiger, Bacteroides and Clostridium XIVa, and when proportions of these genera were solely considered for testing the stratification the classification power increased and the score was nearly excellent (AUC=0.895). While high proportions of Bacteroides were present at baseline in patients with poor clinical benefit (Wilcoxon test, p=0.03363), Faecalibacterium percentages were significantly higher in patients with long-term benefit (p=0.009248; FIG. 2B). Baseline fecal microbiota composition was also significantly associated with patients' overall survival and survival longer than 18 months (Wilcoxon test, p=0.011; FIG. 2C). All patients with an overall survival longer than 18 months could indeed be classified based on their gut microbial composition at V₁ and all showed higher proportions of OTUs affiliated to Firmicutes: Ruminococcus and Lachnospiraceae genus (relatives of Faecalibacterium prausnitzii L2-6, Gemmiger formicilis and butyrate-producing bacterium SS2-1; FIG. 2D). It should be noted that although prescription of antibiotics may alter microbiota composition, antibiotics prior to ipilimumab treatment does not influence baseline dominant microbiota and none of the potentially predictive OTUs or bacterial taxon were associated with antibiotic use.

When the microbiota composition of patients was considered at baseline independently from their clinical characteristics and without a priori hypotheses (k-means clustering algorithm and Calinski-Harabasz index), three groups of patients emerged that were defined as clusters: Cluster A, B and C. The inter-class PCA based on genera composition depending on belonging to any of these 3 clusters highlighted that this stratification was significant (Monte-Carlo test, p=0.00099; FIG. 3A). Microbiota of patients belonging to Cluster A (n=12 at baseline) was driven by Faecalibacterium and other Firmicutes (unclassified Ruminococcaceae, Clostridium XIVa and Blautia). Cluster B (n=10 at baseline) was enriched in Bacteroides, and Cluster C (n=4 at baseline) was enriched in Prevotella (FIG. 3B). Cluster A was associated with longer progression-free survival than Cluster B (p=0.0039; FIG. 3D) and, to a lesser extent, longer overall survival (p=0.051; FIG. 3C). Most of the 22 patients from the Bacteroides-driven Cluster B (80%) received subsequent treatments, including anti-PD-1 agents in 50% of the patients while 58% of patients from the Faecalibacterium-driven Cluster A were treated with subsequent therapies, but only 25% of them received anti-PD-1 agents. Cluster A was also associated with a higher proportion of long-term clinical benefit (8/12; 67%) compared to Cluster B (0/10; p=0.001688, Fisher's exact test).

At the phyla level, microbiota of patients prone to develop colitis was enriched in Firmicutes at baseline (Wilcoxon test, p=0.009), while high proportions of Bacteroidetes was observed in patients who did not develop colitis (p=0.011; FIG. 4A). In competing risk analysis, belonging to Cluster A (driven by Faecalibacterium and other Firmicutes) tended to be associated with a shorter colitis-free cumulative incidence compared to Cluster B (driven by Bacteroides) (Gray's test; p=0.0545, FIG. 4B). Using linear discriminant analysis effect size (LEfSe) analysis, fifteen bacterial OTUs were detected as potential biomarkers either of colitis-free progression or colitis onset during ipilimumab treatment (FIG. 4C). Five (out of six) OTUs from the Bacteroidetes phylum (such as B.uniformis, B. vulgatus and Parabacteroides distasonis) were associated with absence of colitis, whereas OTUs associated with subsequent colitis related mostly to the Firmicutes phylum (8/9 OTUs). Several of these potential colitis-predictive baseline OTUs (relatives of Faecalibacterium prausnitzii L2-6, butyrate producing bacterium L2-21 and Gemmiger formicilis ATCC 27749) were also associated with longer overall survival.

Baseline Immune Parameters Related to Microbiota Clustering, Clinical Benefit and Ipilimumab-Induced Colitis.

Twenty-six patients had both immune monitoring and microbiota composition analysis at V₁. Patients from Cluster C could not be included in this analysis due to the low number of patients belonging to this cluster (n=4). Since patients who belonged to Cluster A (n=12) and Cluster B (n=10) had no differences in absolute counts of peripheral-blood CD4⁺ and CD8⁺ T cells (Table S5), the main T cell subpopulations were considered as a percentage. Patients who belonged to the Faecalibacterium-driven Cluster A had a low proportion of baseline regulatory T cells (Tregs), and had a significantly lower proportion of baseline α4⁺δ7⁺ CD4⁺ (p=0.0447) and α4⁺δ7⁺ (p=0.0344) T cells, compared to those who belonged to the Bacteroides-driven Cluster B (FIG. 5A).

Patients with long-term benefit tended to have a lower frequency of regulatory T cells (p=0.056) and a lower frequency of α4⁺δ7⁺ CD4⁺ (p=0.014) and α4⁺δ7⁺ CD8⁺ (p=0.0081) T cells, compared to those with poor clinical benefit (FIG. 5B). Although serum concentrations of several biomarkers of inflammation (IL-6, IL-8, IP-10, MCP-1, TNFα, sCD25) and sCD14 considered as a marker of bacterial invasion (translocation) were measured, none of these were associated with any cluster or clinical benefit at baseline.

Patients who developed ipilimumab-induced colitis tended to have significantly higher absolute numbers of CD4+ T cells (p=0.0529) and had significantly lower levels of IL-6, IL-8 and sCD25 at baseline compared to patients without colitis (FIG. 5C). In competing risk analysis, patients with high numbers of conventional CD4⁺ T cells had a shorter colitis-free cumulative incidence, compared to those with low numbers of conventional CD4⁺ T cells (p=0.0038). The median percentage of Tregs were lower before introduction of ipilimumab in patients who developed colitis during the course of ipilimumab compared to patients without colitis (2.5% vs 8.4%, p=0.0182). However, absolute numbers of Tregs were not significantly different. None of the other CD4⁺ T cell subpopulations at baseline were related to colitis onset.

The Inducible T-cell COStimulator (ICOS) Molecule is Significantly Up-Regulated on CD4⁺ T Cells after Ipilimumab Treatment in Patients who belong to Faecalibacterium-Driven Cluster A

Since immune parameters were monitored longitudinally, specific changes in immune activation during the course of ipilimumab treatment were assessed in patients from Cluster A vs B. During ipilimumab treatment, Inducible T-cell COStimulator (ICOS) was significantly increased on conventional CD4⁺ T and Treg cells in patients who belonged to Cluster A (FIG. 6A-B), but not in those who belonged to Cluster B (FIG. 6A-B). Given that ICOS is induced on CD4⁺ T cells after IL-2 stimulation, concentration of sCD25, a marker of IL-2 impregnation [29, 30], was surveyed during the course of ipilimumab treatment. Patients who belonged to Cluster A showed a greater increase over time in sCD25 than patients from Cluster B, as demonstrated by a higher V_{2/3} to V₁ ratio compared to patients from Cluster B (FIG. 6C).

III. Conclusion

This study shows that a distinct baseline gut microbiota composition is associated with an anti-cancer response and immune-mediated colitis in patients with metastatic melanoma treated with ipilimumab. Colonization by Firmicutes and more specifically by OTUs related to Faecalibacterium prausnitzii L2-6, butyrate producing bacterium L2-21 and Gemmiger formicilis ATCC 27749, is associated with both anti-cancer response and immune-related colitis. There is a higher representation of Bacteroidetes (mostly Bacteroides genus) in patients who have a poor anti-cancer response and who remain colitis-free.

In addition, in our dataset, Bacteroides fragilis and Bacteroides thetaiotaomicron were not abundantly represented at baseline. Overall, these bacterial species were more abundant in patients with poor clinical benefit who remained colitis-free.

We found that the Faecalibacterium-driven Cluster A and clinical benefit were associated with low baseline percentage of circulating α4⁺δ7⁺ T cells and CD4⁺ Tregs. In addition, high proportions of Faecalibacterium prausnitzii, low baseline percentage of circulating CD4⁺ Tregs and low baseline levels of systemic inflammatory proteins, such as IL-6 IL-8 and sCD25, were associated with subsequent colitis.

It has also been observed that patients who belong to the Faecalibacterium-driven Cluster A have a higher ICOS induction on CD4⁺ T cells and a higher increase in sCD25. Taken together, these observations show that gut microbiota composition at baseline can predict which patients will adequately respond to ipilimumab.

IV. Result of Metabolomics Studies

From the OTUs population described in the overall cohort, metabolomic profiles have been deduced in silico applying the PICRUSt algorithm combined with LEFSe.

A specific set of metabolic pathways (described as KEGG IDs) have been highlighted specifically associated with response to anti CTLA4 (see FIG. 11) or toxicity to anti CTLA4 (see FIG. 10).

High Throughput Metabolomics was Performed on the Fecal Samples at Baseline. Following metabolites extraction, sample were analysed by LC-HRMS as follows:

High-performance liquid chromatographic (HPLC) technique for separation of polar and hydrophilic compounds: aSequant ZIC-pHILIC (Merck, Darmstadt,Germany),

Gradient analysis for 30 minutes.

Q-Exactive mass spectrometry (Thermo Fisher Scientific),

High resolution analyses (70 000 FWHM) alternating positive and negative ionization modes.

For each metabolite (unique m/z & RT pair) identified, ion intensity (peak area) is reported for each sample. Semi-quantitative analysis can be performed comparing the relative intensity of a specific metabolite between samples and data are normalized as relative abundance (proportions) of each metabolite within one sample's profile.

Out of overall 256 detected metabolites, a combination of 14 metabolites can help decipher before aCTLA4 treatment whereas patients are more or less susceptible to answer adequately to the treatment. These 14 metabolites belong to 3 different sets. First, a combination of metabolites can be screened as biomarkers of response to aCTLA4. The LEfSe (linear discriminant analysis effect size) algorithm was applied for high dimensional discovery of biomarkers that discriminate between patients that will respond (or not) to ipilimumab. The algorithm is freely available at http://huttenhower.sph.harvard.edu/galaxy/.

These metabolic biomarkers are more often detected and in higher abundance in fecal samples of the clinical group they belong to.

Main metabolites that help discriminate between Responders and Non Responders are:

1—Trehalose-Sucrose-Isomaltose (p=0.0266)

2—Guanine (p=0.07523)

3—D-Raffinose (p=0.08581)

4—3,4-Dihydroxyphenylacetic acid (p=0.0755)

which are more present in feces of Responders and

5-12-Hydroxydodecanoic acid (p=0.006172)

which is more present in feces of Non responders (see FIG. 12).

Individual repartition of these 5 metabolites in fecal samples of patients is represented in FIG. 6.

A second set of metabolites (n=3), even though not specific of each samples from one clinical group, were significantly differentially represented between patients that responded and patients that did not respond to the aCTLA4 treatment. These metabolites are in Table VII below:

TABLE VII

| Wilcoxon pvalue | Metabolite | Response | Delta |
|---|---|---|---|
| 0.03128 | 1-Methylxanthine | Non Responders | 5x more |
| 0.02251 | Pipecolinic acid | Non Responders | 3x more |
| 0.008023 | 2-Isopropylmalic acid | Responders | 4x more |

Of note, isopropylmalic acid levels were also higher at baseline in patients that developed ipilimumab-associated colitis (1.6× more in patients with colitis)

Finally, a third set of metabolites (n=6) were selected that allowed stratification of the patients that were not properly classified with the previous 2 sets of metabolites. This 3^{rd} set included:

1—Alpha D Amino adipic acid (higher levels in Non Responders)

2—Kynurenic acid (higher levels in Responders)

3—Methylimidazoleacetic acid (higher levels in Non Responders)

4—3, 4 dihydroxyhydrocinnamic acid (higher levels in Non Responders)

5—Cotinine (higher levels in Non Responders)

6—2 Methylnicotinamide (higher levels in Non Responders)

Taken together, these 14 metabolites allow the segregation between responders and non responders at baseline (PCA; component 1 explain 23.64% of variance, and component 2 explains 12.93% of variance; Monte-Carlo randtest test on the inter-class PCA simulated p-value=0.00789).

The ROC curve was built based on these 14 metabolites and the area under the curve showed good prediction value (AUC =0.8602941) (see FIG. 7). While the combination of the 1st set of metabolites (5 selected biomarkers) show good predictive value (AUC>0.8), addition of the Sets #2 and #3 increase stratification power with an AUC>0.85, ameliorating hence the biomarkers specificity (see FIG. 8).

Distribution of the 14 metabolites within the 2 groups of patients is represented in the boxplots (y-axis=peak area) of FIG. 9.

### REFERENCES

1. Robert C, Thomas L, Bondarenko I et al. Ipilimumab plus dacarbazine for previously untreated metastatic melanoma. N. Engl. J. Med. 2011; 364(26):2517-2526.
2. Hodi FS, O'Day S J, McDermott D F et al. Improved Survival with Ipilimumab in Patients with Metastatic Melanoma. N. Engl. J. Med. 2010; 363(8):711-723.
3. Schadendorf D, Hodi F S, Robert C et al. Pooled Analysis of Long-Term Survival Data From Phase II and Phase III Trials of Ipilimumab in Unresectable or Metastatic Melanoma. J. Clin. Oncol. 2015; 33(17):1889-1894.
4. Pardoll D M. The blockade of immune checkpoints in cancer immunotherapy. Nat. Rev. Cancer 2012; 12(4):252-264.
5. Postow M A, Callahan M K, Wolchok J D. Immune Checkpoint Blockade in Cancer

Therapy. J. Clin. Oncol. 2015; 33(17):1974-1982.
6. Weber J S, Kahler K C, Hauschild A. Management of Immune-Related Adverse Events and Kinetics of Response With Ipilimumab. J. Clin. Oncol. 2012;
30(21):2691-2697.
7. Beck K E, Blansfield J A, Tran KQ et al. Enterocolitis in Patients With Cancer After Antibody Blockade of Cytotoxic T-Lymphocyte-Associated Antigen 4. J. Clin. Oncol. 2006; 24(15):2283-2289.

8. Gupta A, De Felice K M, Loftus E V, Khanna S. Systematic review: colitis associated with anti-CTLA-4 therapy. Aliment. Pharmacol. Ther. 2015; 42(4):406-417.
9. Marthey L, Mateus C, Mussini C et al. Cancer Immunotherapy with Anti-CTLA-4 Monoclonal Antibodies Induces an Inflammatory Bowel Disease. ECCOJC 2016; 10(4):395-401.

10. Abraham C, Cho J H. Inflammatory Bowel Disease. N. Engl. J. Med. 2009;

361(21):2066-2078.
11. Chassaing B, Darfeuille-Michaud A. The Commensal Microbiota and Enteropathogens in the Pathogenesis of Inflammatory Bowel Diseases. Gastroenterology 2011; 140(6):1720-1728.e3.
12. Lepage P, Hasler R, Spehlmann M E et al. Twin Study Indicates Loss of Interaction Between Microbiota and Mucosa of Patients With Ulcerative Colitis. Gastroenterology 2011; 141(1):227-236.
13. Vétizou M, Pitt J M, Daillère R et al. Anticancer immunotherapy by CTLA-4 blockade relies on the gut microbiota. Science 2015; 350(6264):1079-1084.
14. Sivan A, Corrales L, Hubert N et al. Commensal Bifidobacterium promotes antitumor immunity and facilitates anti-PD-L1 efficacy. Science 2015; 350(6264):1084-1089.
15. Wolchok J D, Hoos A, O'Day S et al. Guidelines for the evaluation of immune therapy activity in solid tumors: immune-related response criteria. Clin. Cancer Res. Off. J. Am. Assoc. Cancer Res. 2009; 15(23):7412-7420.
16. Snyder A, Makarov V, Merghoub T et al. Genetic basis for clinical response to CTLA-4 blockade in melanoma. N. Engl. J. Med. 2014; 371(23):2189-2199.
17. Seksik P. Alterations of the dominant faecal bacterial groups in patients with Crohn's disease of the colon. Gut 2003; 52(2):237-242.
18. Chen H, Liakou CI, Kamat A et al. Anti-CTLA-4 therapy results in higher CD4⁺ ICOShi T cell frequency and IFN-gamma levels in both nonmalignant and malignant prostate tissues. Proc. Natl. Acad. Sci. U. S. A. 2009; 106(8):2729-2734.
19. Read S, Greenwald R, Izcue A et al. Blockade of CTLA-4 on CD4⁺ CD25⁺ regulatory T cells abrogates their function in vivo. J. Immunol. Baltim. Md 1950 2006; 177(7):4376-4383.
20. Lukiw W J. Bacteroides fragilis Lipopolysaccharide and Inflammatory Signaling in Alzheimer's Disease. Front. Microbiol. 2016; 7:1544.
21. Guigoz Y, Dore J, Schiffrin EJ. The inflammatory status of old age can be nurtured from the intestinal environment. Curr. Opin. Clin. Nutr. Metab. Care 2008; 11(1):13-20.
22. Le Chatelier E, Nielsen T, Qin J et al. Richness of human gut microbiome correlates with metabolic markers. Nature 2013; 500(7464):541-546.
23. Sokol H, Pigneur B, Watterlot L et al. Faecalibacterium prausnitzii is an anti-inflammatory commensal bacterium identified by gut microbiota analysis of Crohn disease patients. Proc. Natl. Acad. Sci. U. S. A. 2008; 105(43):16731-16736.
24. Devillard E, McIntosh FM, Duncan S H, Wallace R J. Metabolism of linoleic acid by human gut bacteria: different routes for biosynthesis of conjugated linoleic acid. J. Bacteriol. 2007; 189(6):2566-2570.
25. Mathewson N D, Jenq R, Mathew A V et al. Gut microbiome-derived metabolites modulate intestinal epithelial cell damage and mitigate graft-versus-host disease. Nat. Immunol. 2016; 17(5):505-513.
26. Smith P M, Howitt M R, Panikov N et al. The microbial metabolites, short-chain fatty acids, regulate colonic Treg cell homeostasis. Science 2013; 341(6145):569-573.
27. Gray R J. A Class of $K$-Sample Tests for Comparing the Cumulative Incidence of a Competing Risk. Ann Stat. 1988; 16(3):1141-1154.
28. Lin G, So Y, Johnston G. Analysing survival data with competing risks using SAS* software. Proc. SAS Glob. Forum 2012 Conf. Cary N C SAS Inst. Inc 2012.
29. Riley J L, Blair P J, Musser J T et al. ICOS Costimulation Requires IL-2 and Can Be Prevented by CTLA-4 Engagement. J. Immunol. 2001; 166(8):4943-4948.
30. Lotze M T, Custer M C, Sharrow S O et al. In vivo administration of purified human interleukin-2 to patients with cancer: development of interleukin-2 receptor positive cells and circulating soluble interleukin-2 receptors following interleukin-2 administration. Cancer Res. 1987; 47(8):2188-2195.
31. Dubin K, Callahan M K, Ren B et al. Intestinal microbiome analyses identify melanoma patients at risk for checkpoint-blockade-induced colitis. Nat. Commun. 2016; 7:10391.
32. Maynard C L, Elson C O, Hatton R D, Weaver C T. Reciprocal interactions of the intestinal microbiota and immune system. Nature 2012; 489(7415):231-241. 33. Cebula A, Seweryn M, Rempala GA et al. Thymus-derived regulatory T cells contribute to tolerance to commensal microbiota. Nature 2013; 497(7448):258-262.
34. Png C W, Linden S K, Gilshenan K S et al. Mucolytic bacteria with increased prevalence in IBD mucosa augment in vitro utilization of mucin by other bacteria. Am. J. Gastroenterol. 2010; 105(11):2420-2428.
35. Hedin C R, McCarthy N E, Louis P et al. Altered intestinal microbiota and blood T cell phenotype are shared by patients with Crohn's disease and their unaffected siblings. Gut 2014; 63(10):1578-1586.
36. Romano E, Kusio-Kobialka M, Foukas P G et al. Ipilimumab-dependent cell-mediated cytotoxicity of regulatory T cells ex vivo by nonclassical monocytes in melanoma patients. Proc. Natl. Acad. Sci. U. S. A. 2015; 112(19):6140-6145.
37. Fu T, He Q, Sharma P. The ICOS/ICOSL Pathway Is Required for Optimal Antitumor Responses Mediated by Anti-CTLA-4 Therapy. Cancer Res. 2011; 71(16):5445-5454.
38. Liakou C I, Kamat A, Tang DN et al. CTLA-4 blockade increases IFN-producing CD4+ICOShi cells to shift the ratio of effector to regulatory T cells in cancer patients. Proc. Natl. Acad. Sci. 2008; 105(39):14987-14992.
39. Sim G C, Martin-Orozco N, Jin L et al. I L-2 therapy promotes suppressive ICOS+ Treg expansion in melanoma patients. J. Clin. Invest. 2014; 124(1):99-110.
40. Klatzmann D, Abbas A K. The promise of low-dose interleukin-2 therapy for autoimmune and inflammatory diseases. Nat. Rev. Immunol. 2015; 15(5):283-294.
41. Chaput N, Flament C, Locher C et al. Phase I clinical trial combining imatinib mesylate and IL-2: HLA-DR(+) NK cell levels correlate with disease outcome. Oncoimmunology 2013; 2(2):e23080.
42. Yu A, Snowhite I, Vendrame F et al. Selective IL-2 responsiveness of regulatory T cells through multiple intrinsic mechanisms supports the use of low-dose IL-2 therapy in type 1 diabetes. Diabetes 2015; 64(6):2172-2183.
43. Hodi F S, Chesney J, Pavlick A C et al. Combined nivolumab and ipilimumab versus ipilimumab alone in patients with advanced melanoma: 2-year overall survival outcomes in a multicentre, randomised, controlled, phase 2 trial. Lancet Oncol. 2016; 17(11):1558-1568.
44. Larkin J, Hodi F S, Wolchok J D. Combined Nivolumab and Ipilimumab or Monotherapy in Untreated Melanoma. N. Engl. J. Med. 2015; 373(13):1270-1271.

1-56. (canceled)

57. A composition comprising one or more purified bacterial strains with 16S rRNA sequences having at least 97% sequence identity with bacterial strains of species selected from the group consisting of Lachnospiraceae butyrate producing bacterium, Bacteroides ovatus, Ruminococcaceae clostridiales bacterium, Blautia obeum, Fusicatenibacter saccharivorans, Roseburia inulinivorans, Gemmiger formicilis, and Faecalibacterium prausnitzii.

58. The composition according to claim 57 comprising one or more purified bacterial strains of species selected from the group consisting of Lachnospiraceae butyrate producing bacterium, Bacteroides ovatus, Ruminococcaceae clostridiales bacterium, Blautia obeum, Fusicatenibacter saccharivorans, Roseburia inulinivorans, Gemmiger formicilis, and Faecalibacterium prausnitzii.

59. The composition according to claim 57 wherein the purified bacterial strains have 16S rRNA sequences having at least 97%, at least 98%, or at least 99% sequence identity.

60. The composition according to claim 57 wherein the composition comprises two or more, three or more, four or more, five or more, six or more, seven or more, eight or more, nine or more, or ten or more bacterial strains.

61. The composition according to claim 57 wherein at least 10%, at least 20%, at least 30%, at least 40%, at least 50%, at least 60%, at least 70%, at least 80%, at least 90%, or at least 100% of the bacterial strains belong to the Firmicutes phylum.

62. The composition according to claim 57 wherein less than 100%, less than 90%, less than 80%, less than 70%, less than 60%, less than 50%, less than 40%, less than 30%, less than 20%, or less than 10%, of the bacterial strains belong to the genus Bacteroides.

63. The composition according to claim 57 wherein the composition does not include bacterial strains of the genus Bacteroides.

64. The composition according to claim 57 wherein the composition does not include bacterial strains of the species Bacteoides fragilis or Bacteoides thetaiotamicron.

65. The composition according to claim 57 wherein the composition does not include bacterial strain Faecalibacterium prausnitzii A2-165.

66. The composition according to claim 57 wherein the bacterial strains are lyophilized.

67. The composition according to claim 57 wherein the composition further comprises an immune checkpoint inhibitor.

68. The composition according to claim 67 wherein the immune checkpoint inhibitor is a PD-1 inhibitor, PD-L1 inhibitor, or CTLA-4 inhibitor.

69. A pharmaceutical composition comprising the composition according to claim 57 and a pharmaceutically acceptable carrier.

70. The pharmaceutical composition according to claim 69, wherein the pharmaceutical composition is formulated for delivery to the intestine.

71. The pharmaceutical composition according to claim 69, wherein the pharmaceutical composition is in the form of a capsule.

72. The pharmaceutical composition according to claim 71, wherein the pharmaceutical composition is formulated for oral administration.

73. The pharmaceutical composition according to claim 69, wherein the pharmaceutical composition comprises a pH sensitive composition comprising one or more enteric polymers.

74. A method of treating cancer comprising administering a pharmaceutically effective amount of the pharmaceutical composition of claim 69 to treat the cancer in the subject.

75. The method of claim 74 wherein the cancer is melanoma.

76. The method of claim 74 further comprising determining if the subject has developed colitis.


SEQUENCE LISTING (34 sequences)

SEQ ID NO: 1 - DNA, 418 bp, Parabacteroides distasonis
tgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtgggggatc

SEQ ID NO: 2 - DNA, 402 bp, uncultured bacterium NA48 AY975552
tggggaatattgcacaatgggggaaaccctgatgcagcgacgccgcgtgagtgaagaagtatttcggtatgtaaagctctatcagcagggaagaaagtgacggtacctgaataagaagccccggctaactacgtgccagcagccgcggtaatacgtagggggcaagcgttatccggatttactgggtgtaaagggagcgtagacggcaaggcaagtctgaagtgaaagcccggtgcttaacgccgggactgctttggaaactgtttggctggagtgccggagaggtaagcggaattcctagtgtagcggtgaaatgcgtagatattaggaagaacaccagtggcgaaggcggcttactggacggtaactgacgttgaggctcgaaagcgtggggagcaaaca

SEQ ID NO: 3 - DNA, 396 bp, butyrate producing bacterium SS2 1 AY305319
tggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggga

SEQ ID NO: 4 - DNA, 430 bp, uncultured bacterium C3 13 GQ896957
tggggaatattgggcaatgggcgcaagcctgacccagcaacgccgcgtgaaggaagaaggctttcgggttgtaaacttcttttgtcggggacgagtagaagacggtacctggcgaataagccacggctaactacgtgccagcagccgcggtaatacgtaggtggcaagcgttgtccggatttactgggtgtaaagggcgtgtagccgggaaggcaagtcagatgtgaaatccacgggcttaactcgtgaactgcatttgaaactacttttcttgagtatcggagaggcaatcggaattcctagtgtagcggtgaaatgcgtagatattaggaggaacaccagtggcgaaggcggattgctgggacgacaactgacgggtgagggcgcgaaagcgtgggggagcaaacagggattagataccctgggtagt

SEQ ID NO: 5 - DNA, 401 bp, Faecalibacterium prausnitzii
tggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggagaacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaac

SEQ ID NO: 6 - DNA, 422 bp, Gemmiger formicilis
tgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttgtccggaattactgggtgtaaagggagcgcaggcggaccggcaagttggaagtgaaaactatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacaggattagataccctggtagt

SEQ ID NO: 7 - DNA, 398 bp, uncultured bacterium RL246 aai74e12 DQ793623
tggggaatattgcacaatgggggaaaccctgatgcagcgacgccgcgtggaggaagaaggtcttcggattgtaaactcctgttgttggggaagataatgacggtacccaacaaggaagtgacggctaactacgtgccagcagccgcggtaaaacgtgggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcctactggggcaccaactgacgctgagggctcgaaagtgtggggta

SEQ ID NO: 8 - DNA, 204 bp, Bacteroides ovatus
acgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgtt

SEQ ID NO: 9 - DNA, 202 bp, Faecalibacterium prausnitzii
acgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtg

SEQ ID NO: 10 - DNA, 202 bp, Eisenbergiella massiliensis
acgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtg

SEQ ID NO: 11 - DNA, 204 bp, uncultured bacterium REC1M 21 AY343160
acgggaggcagcagtggggaatattgggcaatgggcgcaagcctgacccagcaacgccgcgtgaaggaagaaggctttcgggttgtaaacttcttttgtcagggacgagtagaagacggtacctgacgaataagccacggctaactacgtgccagcagccgcggtaatacgtaggtggcaagcgttgtccggatttactgggtg

SEQ ID NO: 12 - DNA, 402 bp, butyrate producing bacterium L2 21 AJ270477
tggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtgggggagcaaac

SEQ ID NO: 13 - DNA, 264 bp, Bacterium uniformis JCM5828 3 EU136680
acggaaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtt

SEQ ID NO: 14 - DNA, 204 bp, Bacteroides sp ALA Bac AM117579
acgggaggcagcagtgaggaatattggtcaatgggcgagagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgtt

SEQ ID NO: 15 - DNA, 204 bp, Bacteroides uniformis JCM 5828T AB50110
acgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgtt

SEQ ID NO: 16 - DNA, 324 bp, Blautia obeum AY169419
tggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagata

SEQ ID NO: 17 - DNA, 1436 bp, Parabacteroides distasonis
tatgaacgctagcgacaggcttaacacatgcaagtcgaggggtagcgggggtagcgatacccgccggcgaccggcgcacgggtgagtaacgcgtatgcaacttgcctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtgggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaggccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcctagggtaaaactg

SEQ ID NO: 18 - DNA, 1422 bp, uncultured bacterium NA48 AY975552
ccttctaatagagtttgatcctggctcaggatgaacgctggcgcctttcctaacacatgcaagtcgaacggagctgctttgatgaagttttcggatggatttaaaacagcttagtggcggacgggtgagtaacgcgtgggtaacctgcctcacactgggggataacagttagaaatagctgctaataccgcataagcgcacagttccgcatggaacagtgtgaaaaactccggtggtgtgagatggacccgcgtctgattagccagttggcggggtaacggcccaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaagtgacggtacctgaataagaagccccggctaactacgtgccagcagccgcggtaatacgtagggggcaagcgttatccggatttactgggtgtaaagggagcgtagacggcaaggcaagtctgaagtgaaagcccggtgcttaacgccgggactgctttggaaactgtttggctggagtgccggagaggtaagcggaattcctagtgtagcggtgaaatgcgtagatattaggaagaacaccagtggcgaaggcggcttactggacggtaactgacgttgaggctcgaaagcgtggggagcaaacaggattagataccctggtagtccacgccgtaaacgatgattgctaggtgtaggtgggtatggacccatcggtgccgcagctaacgcaataagcaatccacctgggggagtacgttcgcaagaatgaaactcaaaggaattgacggggacccgcacaagcggtggagcatgtggtttaattcgaagcaacgcgaagaaccttaccaggtcttgacatcccgatgaaaaacccggtaacggggttccctctttcggagcatccggagacaaggtgggggcatgggtttgccgccagctcctggtcctggaaaattttggggttaaatccccccaaccaaccgcacaccctttattcttttgaaccccacaaggtaaaggcttgggcactcctaagaggaactggcccggggataacccggaggaaggtggggatgacgtcaaatcatcatgccccttatgatctgggctacacacgtgctacaatggcgtaacaaagggaagcgagcctgcgagggtgagcaaatcccaaaaataacgtcccagttcggactgtagtctgcaacccgactacacgaagctggaatcgctagtaatcgcgaatcagaatgtcgcggtgaatacgttcccgggtcttgcacacaccgcccgtc

SEQ ID NO: 19 - DNA, 1491 bp, butyrate producing bacterium SS2 1 AY305319
agagtttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctaccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtgggggagcaaacaggattagataccctggtagtccacgccgtaaacgatgaatactaggtgtcggggccgtagaggctccggtgccgcagccaacgcagtaagtattccacctggggagtacgctcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagtgacaggtggtgcatggttgtcgtcagctcgtgtcgtgagatgttgggttaagtcccgcaacgagcgcaacccctatctttagtagccagcatttcaggtgggcactctagagagactgccagggataacctggaggaaggtggggacgacgtcaaatcatcatgccccttacgaccagggctacacacgtgctacaatggcgtaaacagagggaagcagcctcgtgagagtgagcaaatcccaaaaataacgtctcagttcggattgtagtctgcaactcgactacatgaagctggaatcgctagtaaccgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggt

SEQ ID NO: 20 - DNA, 1488 bp, uncultured bacterium C3 13 GQ896957
ggttcgattctggctcaggacgaacgctggcggcgtgcttaacacatgcaagtcgaacgagaatcactggaaggagttttcggacaacggaaggtgaggacagtggcggacgggtgagtaacgcgtgaggaacctgcctttcagagggggacaacagttggaaacgactgctaataccgcatgacacatagagatcgcatggtttttatgtcaaagatttatcgctgaaagatggcctcgcgtctgattagctagttggtgaggtaacggcccaccaaggcgacgatcagtagccggactgagaggttgaccggccacattgggactgagatacggcccagactcctacgggaggcagcagtggggaatattgggcaatgggcgcaagcctgacccagcaacgccgcgtgaaggaagaaggctttcgggttgtaaacttcttttgtcggggacgagtagaagacggtacctggcgaataagccacggctaactacgtgccagcagccgcggtaatacgtaggtggcaagcgttgtccggatttactgggtgtaaagggcgtgtagccgggaaggcaagtcagatgtgaaatccacgggcttaactcgtgaactgcatttgaaactacttttcttgagtatcggagaggcaatcggaattcctagtgtagcggtgaaatgcgtagatattaggaggaacaccagtggcgaaggcggattgctggacgacaactgacggtgaggcgcgaaagcgtggggagcaaacaggattagataccctggtagtccacgctgtaaacgatgaatactaggtgtgcggggactgaccccctgcgtgccgcagttaacacaataagtattccacctggggagtacgatcgcaaggttgaaactcaaaggaattgacgggggcccgcacaagcggtggattatgtggtttaattcgacgcaacgcgaagaaccttaccagggcttgacatcctactaacgaagtagagatacatcaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggatgcaaaaccgtaaggtggagcgaacccctaaaagccgtctcagttcagattgtgggctgcaacccgcccacatgaagtcggaatcgctagtaatcgcggatcagcatgccgcggtgaatacgttcccgggccttgtacacaccgcccgtcacaccatgagagtcgggaacacccgaagtccgtagcctaaccgtaaggagggcgcggccgaaggtgggttcgataattggggtgaagtcgtaacaaggtaccc

SEQ ID NO: 21 - DNA, 1372 bp, Faecalibacterium prausnitzii
caagtcgaacgagagatgaggagcttgctcttcagatcgagtggcgaacgggtgagtaacgcgtgaggaacctgcctcaaagagggggacaacagttggaaacgactgctaataccgcataagcccacggctcggcatcgagcagagggaaaaggagtgatccgctttgagatggcctcgcgtccgattagctggttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggagaacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacaccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcccttgacgaacatagaaatattttttctcttcggagcaaggagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgtggatcagcatgccacggtgaatacgttcccgggccttgtacacaccgcccgtcacaccatgagagccggggggacccgaagtcggtagtctaaccgcaaggaggac

SEQ ID NO: 22 - DNA, 1426 bp, Gemmiger formicilis
catgcagtcgacggagctagaggagcttgcttttcttggcttagtggcgaacgggtgagtaacgcgtgagtaacctgccctggagtgggggacaacagttggaaacgactgctaataccgcataagcccacgatccggcatcggatcgagggaaaaggattttttcgcttcaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttgtccggaattactgggtgtaaagggagcgcaggcggaccggcaagttggaagtgaaaactatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacaggattagataccctggtagtccatgccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagcgcagagatgcatgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagaagcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtcggtagtctaaccgcaaggaggacgccgccgaagtaaaactggtgattggggtgaagtcgtacagggggtaacc

SEQ ID NO: 23 - DNA, 1471 bp, uncultured bacterium RL246 aai74e12 DQ793623
ggttcgattctggctcaggacgaacgctggcggcgcgcctaacacatgcaagtcgaacgagcgagagagagcttgctttctcgagcgagtggcgaacgggtgagtaacgcgtgaggaacctgcctcaaagagggggacaacagttggaaacgactgctaataccgcataagcccacggctcggcatcgagcagggggaaaaggagcaatccgctttgagatggcctcgcgtccgattagctagttggtgaggtaacggcccaccaaggcaacgatcggtagccagactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttggggaagataatgacggtacccaacaaggaagtgacggctaactacgtgccagcagccgcggtaaaacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtggggtagcaaacaggattagataccctggtagtccacaccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcccttgacaggcatagaaatatgttttctcttcggagcaaggagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgtggatcagcatgccacggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtaccc

SEQ ID NO: 24 - DNA, 1383 bp, Bacteroides ovatus
cagtcgaggggcagcattttagtttgcttgcaactgaagatggcgaccggcgcacgggtgagtaacacgtatccaacctgccgataactccggaatagcctttcgaaagaaagattaataccggatagcatacgaatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgc

SEQ ID NO: 25 - DNA, 1372 bp, Faecalibacterium prausnitzii; identical to SEQ ID NO: 21

SEQ ID NO: 26 - DNA, 1495 bp, Eisenbergiella massiliensis
agagtttgatcctggctcaggatgaacgctggcggcgtgcctaacacatgcaagtcgaacgragttatgcagaggaagttttcggatggaatcggcgtaacttagtggcggacgggtgagtaacgcgtgggaaacctgccctgtaccgggggataacacttagaaataggtgctaataccgcataagcgcacagcytcacatgargcagtgtgaaaaactccggtggtacaggatggtcccgcgtctgattagccagttggcagggtracggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatgacaagccagatgtgaaaacccagggctcaaccctgggactgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccaatgacgtgtccgtaayggggcattctcttcggagcattggagacaggtggtgcatggttgtcgtcagctcgtgtcgtgagatgttgggttaagtcccgcaacgagcgcaacccttatccttagtagccagcaggtaaagctgggcactctagggagactgccggggataacccggaggaaggcggggatgacgtcaaatcatcatgccccttatgatttgggctacacacgtgctacaatggcgtaaacaaagggaagcgagacagtgatgttgagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagcctgtgacctaaccgcaagggaggagcagtcgaaggcaggtctaataactggggtgaagtcgtaacaaggtagccgt

SEQ ID NO: 27 - DNA, 1451 bp, uncultured bacterium REC1M 21 AY343160
gacgaacgctggcggcgtgcttaacacatgcaagtcgaacgagaatctacggatcgaggattcgtccaagtgaagtagaggacagtggcggacgggtgagtaacgcgtgaggaacctgcctttcagagggggacaacagttggaaacgactgctaataccgcatgacacattggagtcgcatggctctgatgtcaaagatttatcgctgaaagatggcctcgcgtctgattagctagttggtgaggtaacggcccaccaaggcgacgatcagtagccggactgagaggttgaccggccacattgggactgagatacggcccagactcctacgggaggcagcagtggggaatattgggcaatgggcgcaagcctgacccagcaacgccgcgtgaaggaagaaggctttcgggttgtaaacttcttttgtcagggacgagtagaagacggtacctgacgaataagccacggctaactacgtgccagcagccgcggtaatacgtaggtggcaagcgttgtccggatttactgggtgtaaagggcgtgtagccgggaaggcaagtcagatgtgaaatccacgggctcaactcgtgaactgcatttgaaactgtttttcttgagtatcggagaggcaatcggaattcctagtgtagcggtgaaatgcgtagatattaggaggaacaccagtggcgaaggcggattgctggacgacaactgacggtgaggcgcgaaagcgtggggagcaaacaggattagataccctggtagtccacgctgtaaacgatgaatactaggtgtgcggggactgaccccctgcgtgccgcagttaacacaataagtattccacctggggagtacgatcgcaaggttgaaactcaaaggaattgacggggacccgcacaagcggtggattatgtggtttaattcgaagcaacgcgaagaaccttaccagggcttgacatcctactaacgagatagagatatgttaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggatgcgataccgcaaggtggagcgaacccctaaaagccgtcccagttcagattgtgggctgcaacccgcccacatgaagtcggaatcgctagtaatcgcagatcagcatgctgcggtgaatacgttcccgggccttgtacacaccgcccgtcacaccatgagagtcgggaacacccgaagcctgtagcctaaccattcggagggcgcagtcgaaggtgggttcgataattggggtg

SEQ ID NO: 28 - DNA, 1447 bp, butyrate producing bacterium L2 21 AJ270477
gctcaggatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacagcatcgcatgatgctgtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtgggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaatcgtaccttctcttcggagcaggagtgacaggtggtgcatggttgtcgtcagctcgtgtcgtgagatgttgggttaagtcccgcaacgagcgcaacccttatctttagtagccagcggtcaggccgggcactctagagagactgccagggataacttggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagctgtcgaaggcaggc

SEQ ID NO: 29 - DNA, 1477 bp, Bacterium uniformis JCM5828 3 EU136680
ctggctcaggatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaactggtgattggggctaagtcgtaacaaggtaacc

SEQ ID NO: 30 - DNA, 1416 bp, Bacteroides sp ALA Bac AM117579
ctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcg

SEQ ID NO: 31 - DNA, 1425 bp, Bacteroides uniformis JCM 5828T AB50110
gtttgatcatggctcaggatgaacgctagtacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaa

SEQ ID NO: 32 - DNA, 1439 bp, Blautia obeum AY169419
agagtttgatcctggctcaggatgaacgctggcggcgtgcttaacacatgcaagtcgaacgggaaatayttyattgaracttcggtsgatttratytawttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctwgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggkrgcatrgcymttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaartcttgacatccsyctgaccgrtcyttaaycggaccttyycttcggrrcagrsgwgacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaag

SEQ ID NO: 33 - DNA, 15 bp, artificial
  synthetic sequence
tacggraggcagcag

SEQ ID NO: 34 - DNA, 20 bp, artificial
  synthetic sequence
ggactaccagggtatctaat